(19) United States
(12) Patent Application Publication
(10) Pub. No.: US 2004/0126794 A1
(43) Pub. Date: Jul. 1, 2004

(54) Detection of susceptibility to autoimmune diseases

(76) Inventors: Teodorica L. Bugawan, Castro Valley, CA (US); Henry A. Erlich, Oakland, CA (US)

Correspondence Address: MORGAN, LEWIS & BOCKIUS, LLP., 3300 HILLVIEW AVENUE, PALO ALTO, CA, 94304, US

(21) Appl. No.: 10/672,794

(22) Filed: Sep. 25, 2003

Related U.S. Application Data
(60) Provisional application No. 60/413,955, filed on Sep. 26, 2002.

(51) Int. Cl. C12Q 1/68; C12P 19/34

(52) U.S. Cl. 435/6; 435/91.2

(57) ABSTRACT

The present invention provides methods and reagents for detecting an individual's increased or decreased risk for type 1 diabetes, also known as, insulin dependent diabetes mellitus (“IDDM”).

The present patent application claims priority under 35 U.S.C. § 119(e) to U.S. provisional application No. 60/413,955, filed Sep. 26, 2002, which is incorporated herein by reference in its entirety.

### 1. FIELD OF THE INVENTION

The present invention relates to methods and reagents for detecting an individual's risk for autoimmune diseases. More specifically, it relates to methods and reagents for detecting an individual's increased or decreased risk for type 1 diabetes.

### 2. BACKGROUND OF THE INVENTION

Type 1 diabetes, also known as, insulin dependent diabetes mellitus (“IDDM”), is a chronic autoimmune disease resulting from the destruction of the insulin producing cells (beta cells) in the pancreatic islets of Langerhans leading to clinically insufficient insulin production and, consequently, to dysregulation of glucose metabolism. Atkinson and Maclaren, 1994, N. Engl. J. Med. 331:1428-36. Type 1 diabetes is typically associated with low C-peptide levels and, in most populations studied, with the presence of autoantibodies to various islet cell autoantigens, notably insulin, GAD-65, and IA-2, a tyrosine kinase. These physical manifestations, namely low C-peptide levels and the presence of autoantibodies to islet cell autoantigens, can be used to diagnose an individual as type 1 diabetic.

Type 1 diabetes, as well as a variety of other autoimmune diseases, have been associated with serologically defined variants of the human leukocyte antigen (“HLA”). HLA typing of large groups of patients with various autoimmune diseases has shown that some HLA alleles occur at significantly higher, or lower frequency in these patients than in the general population. From such studies, the relative risk of developing a disease in individuals who inherit certain HLA alleles has been estimated. For example, a strong association has been identified between the autoimmune disease ankylosing spondylitis and the class I HLA allele B27. Individuals who are HLA-B27-positive have approximately a 90 fold greater chance of developing ankylosing spondylitis than individuals lacking B27.

The HLA genes play an important role in an individual's susceptibility to type 1 diabetes as well as other autoimmune diseases. The HLA loci are located on the short arm of chromosome 6 and contain several genes which encode many different glycoproteins. These glycoproteins have been classified into two categories. The first category, class I products, encoded by the HLA-A, HLA-B, and HLA-C genes, are on the surface of all nucleated cells and function as targets in cytolytic T-cell recognition. The second category, class II products, encoded by the HLA-D region, are involved in cooperation and interaction between cells of the immune system. The class II products appear to be encoded by at least three distinct genes, DR, DQ and DP. For a review article, see Giles et al., 1985, Adv. in Immunol. 37:1-71. The HLA genes are highly polymorphic. In the class II genes, the polymorphisms are primarily encoded by the second exon and in the class I genes, the polymorphisms are encoded primarily in the second and third exons (see Zemmour and Parham, 1991, Immunogenetics 33:310-320), although sequence variation in the fourth exon of class I genes is also known (see Malissen et al., 1982, Proc. Natl. Acad. Sci. USA 79:893-897).

In addition to evidence for linkage to the HLA region, type 1 diabetes has been associated, in many different populations, with specific serologically defined HLA class II alleles, in particular with the serotypes DR3 and DR4. Svejgaard et al., 1983, Immunol Rev 70:193-218; Tiwari and Terasaki, 1985, HLA and Disease Associations, Springer-Verlag, NY; Rotter, 1981, Am. J. Hum. Genet. 33:835-851.

The HLA allele frequency distributions as well as their patterns of linkage disequilibrium vary significantly from population to population. The incidence as well as the physical manifestations of the disease differ in the different populations. Type 1 diabetes is less frequent in Asians than among populations in the U.S. and amongst populations originating in Europe. For example, in Japan and China the incidence is about 1:100,000/yr compared to between 18 and 40:100,000/yr in the U.S. or northern Europe. Medici et al., 1999, Diabetes Care 9: 1458-62. In the Philippines, the frequency of type 1 diabetes is thought to be low although accurate estimates of prevalence are not available. Further, among some Asian populations, in addition to the serotypes DR3 and DR4 which are associated with type 1 diabetes in many populations, the serotype DR9 has also been associated with type 1 diabetes. Hu et al., 1993, Human Immunology 38:105-114; Ju et al., 1991, Tissue Antigen 37:218.

Although several specific class II HLA alleles have been associated, either positively or negatively, with type 1 diabetes, because disease associations differ in different populations and races, there is a need to identify more disease-associated alleles as well as disease-associated alleles which are not class II HLA alleles. Identification of new disease-associated alleles will help refine existing methods of detecting an individual's risk for an autoimmune disease such as type 1 diabetes, and will result in a more accurate determination of an individual's risk.

Further, current serologic methods for detecting class I HLA gene polymorphisms are not capable of detecting much of the variation detectable by DNA-based typing methods, and consequently fail to detect the HLA molecules that are actually disease associated. This is because a single serologically defined allele may actually consist of a family of related alleles that differ slightly from one another in their polymorphic residues. Such differences can be identified only by more detailed molecular studies, such as nucleotide sequencing or other DNA-based typing methods.

### 3. SUMMARY OF THE INVENTION

The present invention provides methods for detecting an individual's increased or decreased risk for an autoimmune disease such as type 1 diabetes, also known as, insulin-dependent diabetes mellitus (“IDDM”). The present invention also provides kits, reagents and arrays useful for detecting an individual's risk for autoimmune diseases such as type 1 diabetes.

In one aspect, the present invention provides a method for detecting an individual's increased risk for an autoimmune disease such as type 1 diabetes by detecting the presence of a type 1 diabetes-associated predisposing HLA-C allele in a nucleic acid sample of the individual, wherein the presence of said allele indicates the individual's increased risk for type 1 diabetes.

The individual can belong to any race or population. In one embodiment, the individual is an Asian, preferably a Filipino.

The nucleic acid sample can be obtained from any part of the individual's body, including, but not limited to hair, skin, nails, tissues or bodily fluids such as saliva, blood, etc. The nucleic acid sample can, but need not, be amplified by any amplification method including, but not limited to, polymerase chain reaction (“PCR”).

The predisposing allele can be any predisposing allele in the HLA-C locus. In one embodiment of the invention, the predisposing allele can be any allele identified as predisposing by methods taught herein. In a preferred embodiment, the predisposing allele can be HLA-C*0102 or HLA-C*0302.

The predisposing allele can be detected by any method known in the art for detecting the presence of a specific allele. These methods include, but are not limited to, contacting the nucleic acid sample with one or more nucleic acid molecules that hybridize under stringent hybridization conditions to one or more polymorphisms associated with said allele and detecting the hybridized nucleic acid molecule or molecules, detection by amplification of the nucleic acid sample by, for example, PCR, and by direct sequencing of the nucleic acid sample.

In another aspect, the present invention provides a method for detecting an individual's decreased risk for an autoimmune disease such as type 1 diabetes by detecting the presence of a type 1 diabetes-associated protective class I HLA allele in a nucleic acid sample of the individual, wherein the presence of said allele indicates the individual's decreased risk for type 1 diabetes.

As discussed above, the individual can belong to any race or population. In a preferred embodiment, the individual is an Asian, preferably a Filipino. As also discussed above, the nucleic acid sample can be obtained from any part of the individual's body, and can, but need not, be amplified by methods such as PCR.

The protective allele can be any protective allele in the HLA-A or HLA-C loci. In one embodiment of the invention, the protective allele can be any allele identified as protective by methods taught herein. In a preferred embodiment, the protective allele can be HLA-A*1101, HLA-C*0702 or HLA-C*1502.

Any method known in the art for detecting the presence of a specific allele can be used. These methods include, but are not limited to, those discussed above.

Another aspect of the invention relates to a kit useful for detecting the presence of a predisposing or a protective class I HLA allele in a nucleic acid sample of an individual whose risk for type 1 diabetes is being assessed. The kit can comprise one or more polynucleotides capable of detecting a predisposing or protective class I HLA allele as well as instructions for their use to detect susceptibility for an autoimmune disease such as type 1 diabetes. In preferred embodiments, the polynucleotide or polynucleotides each individually comprise a sequence that hybridizes under stringent hybridization conditions to a nucleic acid sequence in a type 1 diabetes-associated class I HLA-A or -C allele, wherein said nucleic acid sequence comprises one or more polymorphisms associated with said allele. In some embodiments, the polynucleotide or polynucleotides each individually comprise a sequence that is fully complementary to a nucleic acid sequence in a type 1 diabetes-associated class I HLA-A or -C allele, wherein said nucleic acid sequence comprises one or more polymorphisms associated with said allele.

In some embodiments, the polynucleotide can be used to detect the presence of a type 1 diabetes-associated class I HLA allele by hybridizing to the allele under stringent hybridizing conditions. In some embodiments, the polynucleotide can be used as an extension primer in either an amplification reaction such as PCR or a sequencing reaction, wherein the type 1 diabetes-associated class I HLA allele is detected either by amplification or sequencing.

In certain embodiments, the kit can further comprise amplification or sequencing primers which can, but need not, be sequence-specific. The kit can also comprise reagents for labeling one or more of the polynucleotides, or comprise labeled polynucleotides. Optionally, the kit can comprise reagents to detect the label.

In some embodiments, the kit can comprise one or more polynucleotides that can be used to detect the presence of two or more predisposing or protective class I HLA alleles or combinations of predisposing alleles, protective alleles or both.

In another aspect, the invention provides an array useful for detecting the presence of a predisposing or a protective class I HLA allele in a nucleic acid sample of an individual whose risk for type 1 diabetes is being assessed. The array can comprise one or more polynucleotides capable of detecting a predisposing or protective class I HLA allele. The polynucleotides can be immobilized on a substrate, e.g., a membrane or glass. In preferred embodiments, the polynucleotide or polynucleotides each individually comprise a sequence that can hybridize under stringent hybridization conditions to a nucleic acid sequence in a type 1 diabetes-associated class I HLA-A or -C allele, wherein said nucleic acid sequence comprises one or more polymorphisms associated with said allele. In some embodiments, the polynucleotide or polynucleotides each individually comprise a sequence that is fully complementary to a nucleic acid sequence in a type 1 diabetes-associated class I HLA-A or -C allele, wherein said nucleic acid sequence comprises one or more polymorphisms associated with said allele. The polynucleotide or polynucleotides can, but need not, be labeled. In some embodiments, the array can be a micro-array.

In some embodiments, the array can comprise one or more polynucleotides used to detect the presence of two or more predisposing or protective class I HLA alleles or combinations of predisposing alleles, protective alleles or both.

The methods and reagents of the invention can be used to refine the existing methods of detecting an individual's risk for type 1 diabetes. They can also be used diagnostically to detect an individual's risk for type 1 diabetes. The advantages of the methods and reagents of the invention go beyond providing more type 1 diabetes-associated alleles that can be used for detecting risk for type 1 diabetes, to providing new class I HLA alleles that can be used to analyze populations that could not be analyzed with the hitherto known, largely class II HLA alleles.

### 4. BRIEF DESCRIPTION OF THE TABLES

Table 1 provides HLA-A allele frequencies in Filipino patients and controls;

Table 2 provides a test of heterogeneity among A*24 allele frequencies in Filipino patients and controls;

Table 3 provides HLA-C allele frequencies in Filipino patients and controls;

Table 4 provides two-point HLA Class I and DRB1 haplotypes in significant positive disequilibrium in the Filipino control population (2N=188);

Table 5 provides a summary of tests of HLA two-locus haplotypes on Type I diabetes in Filipinos;

Table 6 provides stratification tests of the influence of specific DRB1 alleles on the risk associated with A*1101, A*2402 and C*1502 for type 1 diabetes in Filipinos;

Table 7 provides polynucleotides for the detection of the HLA-C*0102 allele;

Table 8 provides polynucleotides for the detection of the HLA-C*0302 allele;

Table 9 provides polynucleotides for the detection of the HLA-A*1101 allele;

Table 10 provides polynucleotides for the detection of the HLA-C*0702 allele; and

Table 11 provides polynucleotides for the detection of the HLA-C*1502 allele.

### 5. DETAILED DESCRIPTION OF THE PREFERRED EMBODIMENTS

The present invention provides methods, reagents and kits for detecting an individual's increased or decreased risk for an autoimmune disease. Examples of autoimmune diseases include, but are not limited to, multiple sclerosis, myasthenia gravis, Crohn's disease, ulcerative colitis, primary biliary cirrhosis, insulin-dependent diabetes mellitus, Grave's disease, autoimmune hemolytic anemia, pernicious anemia, autoimmune thrombocytopenia, vasculitides such as Wegener's granulomatosis, Behcet's disease, rheumatoid arthritis, systemic lupus erythematosus (lupus), scleroderma, spondyloarthropathies such as ankylosing spondylitis, psoriasis, dermatitis herpetiformis, pemphigus vulgaris and vitiligo. In certain embodiments, the autoimmune disease is type 1 diabetes, also known as insulin-dependent diabetes mellitus (“IDDM”).

Type 1 diabetes is a chronic autoimmune disease characterized by clinically insufficient insulin production and, consequently, dysregulation of glucose metabolism. Type 1 diabetes is typically associated with low C-peptide levels and, in most populations, with the presence of autoantibodies to various islet cell autoantigens, notably insulin, GAD-65, and IA-2.

5.1 Abbreviations

The abbreviations used throughout the specification to refer to nucleic acids comprising specific nucleobase sequences are the conventional one-letter abbreviations. Thus, when included in a nucleic acid, the naturally occurring encoding nucleobases are abbreviated as follows: adenine (A), guanine (G), cytosine (C), thymine (T) and uracil (U). Also, unless specified otherwise, nucleic acid sequences that are represented as a series of one-letter abbreviations are presented in the 5′->3′ direction.

5.2 Definitions

As used herein, the following terms shall have the following meanings:

Two sequences are “complementary” when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence.

The terms “polynucleotide,” “oligonucleotide” and “nucleic acid” have the same meaning and can be used interchangeably throughout. For convenience, and in order to distinguish the nucleic acid sample and the HLA alleles in the sample from the oligonucleotide sequences used to detect them, a DNA or RNA molecule present in an individual or an individual's sample is referred to as a nucleic acid molecule and a DNA or RNA oligonucleotide sequence is referred to as polynucleotide.

“Class I HLA Loci” or “Class I HLA Genes” refers to an approximately 2000 kilobase region of the human major histocompatibility complex genes located on the short arm of chromosome 6 comprising the genes for HLA-A, HLA-B, HLA-C as well as other genes, some of which are well characterized (e.g., HLA-E, HLA-F, HLA-G etc.) and others which are not so well characterized.

“Positively Associated Alleles” include alleles whose frequencies are increased in individuals with the disease relative to individuals without the disease.

“Negatively Associated Alleles” include alleles whose frequencies are decreased in individuals with the disease relative to individuals without the disease.

“Predisposing Alleles” include alleles which are positively associated with an autoimmune disease such as type 1 diabetes. The presence of a predisposing allele in an individual indicates that the individual has an increased risk for the disease relative to an individual without the allele.

“Protective Alleles” include alleles which are negatively associated with an autoimmune disease such as type 1 diabetes. The presence of a protective allele in an individual indicates that the individual has a decreased risk for the disease relative to an individual without the allele.

“Linkage Disequilibrium” (“LD”) refers to alleles at different loci that are not associated at random, i.e., not associated in proportion to their frequencies. If the alleles are in positive linkage disequilibrium, then the alleles occur together more often than expected assuming statistical independence. Conversely, if the alleles are in negative linkage disequilibrium, then the alleles occur together less often than expected assuming statistical independence.

“Odds Ratio” (“OR”) refers to the ratio of the odds of the disease for individuals with the marker(s) (allele(s)) relative to the odds of the disease in individuals without the marker(s) (allele(s)).

“A*1101” refers to an allele (IMGT/HLA Accession Nos. HLA00043 and HLA01037) in the HLA-A locus. IMGT/HLA is part of the international ImMunoGeneTics project (IMGT) and is a database for sequences of the human major histocompatibility complex (referred to as HLA). The IMGT/HLA database includes all the official sequences for the WHO HLA Nomenclature Committee For Factors of the HLA System. The database is maintained by the Anthony Nolan Research Institute in collaboration with the European Bioinformatics Institute.

“C*0102” refers to an allele (IMGT/HLA Accession No. HLA00401) in the HLA-C locus.

“C*0302” refers to an allele (IMGT/HLA Accession Nos. HLA00410 and HLA01543) in the HLA-C locus.

“C*0702” refers to an allele (IMGT/HLA Accession Nos. HLA00434 and HLA01326) in the HLA-C locus.

“C*1502” refers to an allele (IMGT/HLA Accession Nos. HLA00467 and HLA01081) in the HLA-C locus.

“Stringent” as used with reference to hybridization and wash conditions generally refers to conditions that are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 50° C. As other factors may significantly affect the stringency of hybridization, including, among others, base composition, length of the nucleic acid strands, the presence of organic solvents, the extent of base mismatching, the combination of parameters is more important than the absolute measure of any one.

5.3 Method for Detecting Increased or Decreased Risk for Autoimmune Diseases

The present invention provides methods for detecting an individual's increased or decreased risk to an autoimmune disease. The methods of the invention can be applied to any autoimmune disease, including, but not limited to, those listed above. In certain embodiments, the invention provides methods for detecting an individual's increased or decreased risk to type 1 diabetes. In one aspect, the method can comprise the steps of: (a) obtaining a nucleic acid sample from an individual (b) detecting the presence of predisposing or protective or both alleles in the sample; and (c) assessing the individual's risk for the autoimmune disease based on the alleles detected in said individual's nucleic acid sample.

5.3.1 The Individuals

The method described herein can be used to detect increased or decreased risk for autoimmune diseases such as type 1 diabetes in an individual from any race or population. In one embodiment, the method individual is from an Asian population, preferably a Filipino population.

5.3.2 Nucleic Acid Sample

The nucleic acid sample can be any nucleic acid of the individual. The nucleic acid sample can comprise, for instance, DNA or RNA. In certain embodiments, the nucleic acid sample can comprise DNA. The DNA in the sample can be genomic DNA or cloned DNA or cDNA, reverse transcribed from the individual's RNA. The nucleic acid can be single-stranded or double-stranded.

The nucleic acid sample can be obtained from any part of the individual's body, including, but not limited to hair, skin, nails, tissues or bodily fluids such as saliva, blood, sputum and other lung fluids, etc. In certain embodiments, a nucleic acid sample from amniotic fluid of a mother can be used to detect an unborn child's risk for type 1 diabetes. A variety of techniques for extracting nucleic acids from biological samples are known in the art. For example, see the techniques described in Sambrook et al., 1989, Molecular Cloning: A Laboratory Manual, Cold Spring Harbor Laboratory, 1989, 2^{nd} ed., NY; id., 3^{rd} ed., 2001.

The quantity and concentration of nucleic acid for use in the method can vary, and will be apparent to those of skill in the art. In one embodiment, about 10 ng to about 500 ng of nucleic acid can be used. In other embodiments, about 25 ng to about 200 ng of DNA or RNA can be used. In other embodiments, about 50 ng to about 200 ng or about 50 ng to about 100 ng of DNA or RNA can be used. In a preferred embodiment, about 50 ng to about 100 ng of DNA can be used optionally followed by amplification.

In certain embodiments, the nucleic acid obtained can be amplified by methods such as the polymerase chain reaction (“PCR”). The PCR process enables one to amplify a specific sequence of nucleic acid starting from a very small amount of a complex mixture of nucleic acids and is more fully described in U.S. Pat. Nos. 4,683,195; 4,683,202; 4,889,818 and 4,965,188 and European Patent Publication Nos. 237,362 and 258,017, each of which is incorporated herein by reference. Conveniently, PCR primers can contain restriction enzyme recognition sequences so that amplified DNA can be cloned directly into sequencing vectors in order to determine the nucleotide sequence of the amplification product. Scharf et al., 1986, Hum. Immunol. 233:1076, which is incorporated herein by reference. Amplification of the nucleic acid can occur prior to or concurrent with detection of an allele (see infra).

5.3.3 Predisposing Alleles

Predisposing alleles include alleles which are positively associated with an autoimmune disease such as type 1 diabetes and correlate with an increased risk for the disease. The presence of a predisposing allele in an individual indicates that the individual has an increased risk for the disease relative to an individual without the allele.

Alleles that are predisposing to type 1 diabetes can be found in the class I HLA loci, including the HLA-C gene. Examples of predisposing alleles include, but are not limited to, HLA-C*0102 and HLA-C*0302. Other predisposing alleles are described below.

Other useful predisposing alleles can be identified and utilized in the methods described herein. To begin the identification of such alleles, one can, for example, select two groups of individuals, one group with individuals affected with a disease, for example, type 1 diabetes, and the other group with non-diseased (“normal”) individuals without a family history for the disease. The individuals, as described above in Section 5.3.1, can be from any race or population. Preferably the normal individuals are from the same race or population as the individuals with the disease, i.e., for example, the diabetic individuals.

Identification of the two groups of individuals can be followed by determining the alleles of one or more loci present in both groups of individuals by any method known in the art for determining alleles. For example, the alleles of a candidate HLA locus can be determined by HLA typing individuals. Any known method for HLA typing can be used. In one embodiment, HLA typing of individuals in both groups can be carried out so as to identify all the HLA alleles of one or more HLA loci present in the individuals. Any method known in the art for HLA typing, for example, the method described in Example 1, can be used. U.S. Pat. Nos. 4,582,788; 5,110,920; 5,310,893; 5,451,512; 5,541,065; 5,550,039; and 5,567,809, each of which is incorporated herein by reference, also describe methods that can be used for HLA typing of any HLA locus.

Once the alleles of one or more loci have been identified, the distribution of the alleles in the groups can be compared by any method known in the art for carrying out such comparisons. One such method includes, but is not limited to, carrying out the comparisons with “2 by k” tests for heterogeneity, using the log likelihood ratio test or G statistic (see Sokal and Rohlf, 1995, Biometry W.H. Freeman, San Francisco), where k is the number of allele, haplotype or genotype categories under consideration.

Optionally, any statistically significant difference between the distribution of alleles in the two groups can be determined by methods known in the art. In one embodiment, P values can be used to determine the statistical significance of the measurement, such that the smaller the P value, the more significant the measurement (see Example 1). Preferably the P values will be less than 0.05.

In certain embodiments, whether an allele is predisposing or not can be determined from differences in frequency of occurrence of that allele between the two groups of individuals. Any method known in the art for calculating the risk conferred by an allele may be used. One such method includes, but is not limited to, calculating odds ratios to determine which alleles are predisposing. Odds ratios can be calculated by any method known to one of skill in the art and can be used to indicate the direction and magnitude of significant differences between diseased, e.g., a diabetic and normal individuals. An odds ratio of more than 1 can indicate a predisposing allele of the invention. The greater the odds ratio, the more predisposing the allele can be.

Optionally, the effect of haplotypes with and without other alleles to which a predisposing allele may be linked can be compared. An allele could appear predisposing because it is strongly linked to another predisposing allele. A comparison of haplotypes can be carried out in order to exclude such a possibility. In one embodiment, the comparison can be carried out by determining the odds ratio for a particular allele in the presence as well as in the absence, of other alleles to which the allele is linked.

Haplotype frequencies can be estimated for alleles from the diabetic and normal individuals separately by any method known in the art, including, but not limited to, the use of an EM algorithm as seen in Long et al., 1995, Am. J. Hum. Genet., 56:779-810. The estimated haplotype frequencies can be used to calculate linkage disequilibrium (“LD”) values. The haplotypes used can be, but are not limited to, two locus haplotypes. Some of the observed disease associations can be attributed to LD with high risk haplotypes while others cannot.

5.3.4 Protective Alleles

Protective alleles include alleles which are negatively associated with an autoimmune disease such as type 1 diabetes and correlate with a decreased risk for the disease. The presence of a protective allele in an individual indicates that the individual has a decreased risk for the disease relative to an individual without the allele.

Alleles that are protective to type 1 diabetes can be found in the class I HLA loci, including the HLA-A and HLA-C genes. Examples of protective alleles include, but are not limited to, HLA-A*101, HLA-C*0702 and HLA-C*1502. Other protective alleles are described below.

Other useful protective alleles can be identified and utilized in the methods described herein. To begin the identification of such alleles, one can, for example, select two groups of individuals, one group with individuals affected with a disease, for example, type 1 diabetes, and the other with normal individuals without a family history for the disease, as described above.

Identification of the two groups of individuals can be followed by identifying the alleles of one or more loci present in both groups of individuals by any method known in the art for identifying alleles, as described above for predisposing alleles.

Once the alleles of one or more loci have been identified, the distribution of the alleles in the two groups can be compared by any method known in the art for carrying out such comparisons, as described above.

Optionally, whether any statistically significant difference between the distribution of alleles in the two groups can be determined by methods known in the art. In one embodiment, P values may be used to determine the statistical significance of the measurement, as described above.

In certain embodiments, whether an allele is protective or not can be determined from the differences in the frequency of occurrence of that allele between the two groups of individuals. Any method known in the art for calculating the protection conferred by an allele can be used. Such methods include, but are not limited to, calculating odds ratios to determine which alleles are protective, as described above. An odds ratio of less than 1 can indicate a protective allele of the invention. The smaller the odds ratio, the more protective the allele can be.

Optionally, the effect of haplotypes with and without other alleles to which a protective allele may be linked can be compared. An allele could appear protective because it is strongly linked to another protective allele. In one embodiment, the comparison can be carried out by determining the odds ratio for a particular allele in the presence, as well as in the absence, of other alleles to which the allele is linked, as discussed above.

5.3.5 Detecting the Presence of Predisposing or Protective Alleles

In order to detect an individual's risk for type 1 diabetes, predisposing alleles or protective alleles or both in a nucleic acid sample of the individual can be detected by any means known in the art for detecting the presence of an allele. Such methods include, but are not limited to, restriction-fragment-length-polymorphism detection based on allele-specific restriction-endonuclease cleavage (Kan and Dozy, 1978, Lancet ii:910-912), mismatch-repair detection (Faham and Cox, 1995, Genome Res 5:474-482), binding of MutS protein (Wagner et al., 1995, Nucl Acids Res 23:3944-3948), denaturing-gradient gel electrophoresis (Fisher et al., 1983, Proc. Natl. Acad. Sci. U.S.A. 80:1579-83), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S.A. 92:87-91) cleavage of heteroduplex DNA, methods based on allele-specific primer extension (Syvänen et al., 1990, Genomics 8:684-692), genetic bit analysis (Nikiforov et al., 1994, Nucl Acids Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids Res 23:675-682), radioactive or fluorescent DNA sequencing using standard procedures well known in the art, and peptide nucleic acid (PNA) assays (Orum et al., 1993, Nucl. Acids Res. 21:5332-5356; Thiede et al., 1996, Nucl. Acids Res. 24:983-984).

Preferred methods of detecting the presence of a type 1 diabetes-associated predisposing or protective allele in a nucleic acid sample include, but are not limited to, contacting the nucleic acid sample with one or more polynucleotides that hybridize under stringent hybridization conditions to one or more polymorphisms associated with said allele and detecting the hybridized nucleic acid molecule or molecules. The oligonucleotides can, but need not, be immobilized. Other preferred methods include detecting an amplicon from an amplification reaction, for example, the polymerase chain reaction (“PCR”), allele-specific PCR and sequencing the individual's nucleic acid. Some of the above methods are described in greater detail below.

5.3.5.1 Hybridization With One or More Nucleic Acid Molecules

In certain embodiments, one or more polynucleotides that hybridize under stringent hybridization conditions to a particular allele can be used to detect the presence of that allele. One or more polynucleotides can be used to detect the presence of an allele by, for example, stringently hybridizing the polynucleotide to a sequence that comprises one or more polymorphisms associated with the allele and detecting the hybridization.

In certain embodiments, one or more of the polynucleotides can be contacted with a nucleic acid sample of an individual, whose risk for type 1 diabetes is being detected, under conditions that ensure stringent hybridization. Conditions required to ensure stringent hybridization are well known in the art, and are described, for example, in Sambrook et al., supra and Ausubel et al., 1994, Current Protocols in Molecular Biology, Greene Publishing Associates and Wiley Interscience, NY. The polynucleotide or polynucleotides can hybridize to a sequence in the allele that comprises one or more polymorphisms associated with the allele. This hybridization can then be detected by methods known to one of skill in the art. Examples of such detection methods are provided below. In some embodiments, the polynucleotide or polynucleotides can be immobilized on a support, for example, in an array.

The polynucleotide or polynucleotides used in this embodiment can be prepared using any suitable method known in the art. These methods include, but are not limited to, synthesis of a polynucleotide from nucleoside derivatives performed in solution or on a solid support. The synthesis could follow the phosphotriester method (see Narang, et al., 1979, Meth. Enzymol., 68:90; U.S. Pat. No. 4,356,270), or the phosphodiester method (see Brown, et al., 1979, Meth. Enzymol., 68:109). Automated embodiments of these methods could also be employed, for example, by using diethylphosphoramidites as starting materials (see Beaucage et al., 1981, Tetrahedron Letters, 22:1859-1862). Alternatively, the polynucleotide could be synthesized on a modified solid support as described in U.S. Pat. No. 4,458,066. It is also possible to use a polynucleotide which has been isolated from a biological source.

5.3.5.1.1 Hybridization of One or More Polynucleotides to Immobilized Sample

In certain embodiments of the invention, one or more polynucleotides that hybridize under stringent hybridization conditions to a particular allele can be used to detect the presence of a type 1 diabetes-associated allele by hybridization to an immobilized nucleic acid sample of an individual whose risk for type 1 diabetes is being detected. In this embodiment, the nucleic acid sample can be immobilized on any surface, for example, on one or more membranes. The polynucleotides or polynucleotides can be brought in contact with the immobilized nucleic acid sample under conditions that ensure stringent hybridization, as discussed above. In certain embodiments, the polynucleotide can be labeled and the presence of the label on the surface on which the nucleic acid sample is immobilized can indicate the presence of the allele for which the detected nucleic acid molecule or molecules are specific.

One example of such a technique is “dot blot hybridization.” In this technique, the sample containing the individual's nucleic acid can be immobilized on one or more membranes and each membrane can be hybridized with a different labeled polynucleotide that hybridizes with a sequence in the allele that comprises one or more polymorphisms associated with the allele. The sample can be immobilized on the membrane by any method known in the art for immobilizing samples on membranes, one example of which is called “spotting,” and is described by Kafotos et al., 1979, Nucleic Acids Research 7:1541-1552. After hybridization to one or more of the polynucleotides, the sample can be washed to remove unhybridized nucleic acid molecules using suitable methods known in the art. The label can then be detected by using any detection technique, examples of which are discussed below.

In certain embodiments, the polynucleotide or polynucleotides can be labeled with a suitable detectable label moiety, which can be detected by spectroscopic, photochemical, biochemical, immunochemical or chemical methods. The detectable label can be any label that is capable of generating a signal that can be detected by methods known to those of skill in the art. Immunochemical methods include antibodies which are capable of forming a complex with the nucleic acid molecule or molecules under suitable conditions followed by detection of the complex. Biochemical methods include polypeptides or lectins capable of forming a complex with the nucleic acid molecule or molecules under the appropriate conditions followed by detection of the complex.

The detectable label can be linked to any portion of the polynucleotide known to those of skill in the art to be suitable for such a linkage. For instance, the label can be linked to the backbone of the polynucleotide or to a nucleobase. The detectable label can be linked to the polynucleotide by any method known to those of skill in the art. For instance, the detectable label can be linked covalently, either directly or by way of an optional linker, or non-covalently. The optional linker can be any molecule used by those of skill in the art to link two moieties.

Examples of moieties that can be used to label a polynucleotide include, but are not limited to, fluorescent dyes, electron-dense reagents, enzymes (as commonly used in ELISAs), radioactive atoms, metal-ligand charge transfer complexes, biotin, or haptens and proteins for which antisera or monoclonal antibodies are available. A labeled polynucleotide of the invention can be synthesized and labeled using the techniques known to one of skill in the art. For example, a dot-blot assay can be carried out using probes labeled with biotin, as described in Levenson and Chang, 1989, in PCR Protocols: A Guide to Methods and Applications (Innis et al., eds., Academic Press. San Diego), pages 99-112, incorporated herein by reference.

Among radioactive atoms, ³²P is preferred. Methods for introducing ³²P into a nucleic acid molecule or a polynucleotide are known in the art, and include, for example, 5′ labeling with a kinase, or random insertion by nick translation. If biotin is used as the label, a spacer arm can be utilized to attach it to the polynucleotide. Examples of enzymes that can be used include, but are not limited to, HRP and alkaline phosphatase. Suitable fluorescent moieties include, for example, fluorescein, rhodamine, cy dyes, and other fluorescent moieties known to those of skill in the art. Suitable metal-ligand charge transfer complexes include Ru, Os, Re and other metal-ligand charge transfer complexes known to those of skill in the art. Preferably, the label used is non-radioactive. It should be understood that the same label may serve in several different modes. For example, ¹²⁵I may serve as a radioactive label or as an electron-dense reagent. HRP may serve as enzyme or as antigen for a monoclonal antibody. Further, one may combine various labels for desired effect. For example, one might label a probe with biotin, and detect its presence with avidin labeled with ¹²⁵I, or with an anti-biotin monoclonal antibody labeled with HRP. Other permutations and possibilities will be readily apparent to those of ordinary skill in the art, and are considered as equivalents within the scope of the instant invention.

Detection of the hybridized labeled polynucleotide can be accomplished conveniently by a variety of methods and may be dependent on the source of the label or labels employed. For example, a fluorescently labeled nucleic acid molecule can be detected by laser induced fluorescence or by any other technique known to those of skill in the art for detecting a fluorescently labeled molecule. In some embodiments, one or more biotinylated polynucleotides which hybridize under stringent hybridization conditions to the immobilized nucleic acid sample can be detected by first binding the biotin to avidin-horseradish peroxidase (A-HRP) or streptavidin-horseradish peroxidase (SA-HRP), which is then detected by carrying out a reaction in which the HRP catalyzes a color change of a chromogen. A polynucleotide labeled with other groups can be detected by corresponding methods known to those of skill in the art.

Whatever the method for detecting the labeled nucleic acid molecule and determining which nucleic acid molecule of the invention hybridizes under stringent hybridization conditions to class I HLA allelic sequences in the nucleic acid sample, the central feature of the method involves the identification of the class I HLA allele or alleles present in the sample by detecting the variant sequences present.

5.3.5.1.2 Hybridization of the Sample to Immobilized Polynucleotide or Polynucleotides

In another embodiment of the invention, one or more immobilized polynucleotide or polynucleotides can be used to detect the presence of a type 1 diabetes-associated allele by hybridization to a nucleic acid sample of an individual whose risk for type 1 diabetes is being detected. The hybridization can take place with the nucleic acid sample itself or, with an amplified nucleic acid from the sample. According to this method, the polynucleotide or polynucleotides can be immobilized on any surface, for example, on membranes or chips. In some embodiments, the nucleic acid sample or amplified nucleic acid from the sample can be brought in contact with an array comprising one or more polynucleotides each individually comprising a sequence that hybridizes under stringent hybridization conditions to a nucleic acid sequence in a type 1 diabetes-associated class I HLA allele, wherein said nucleic acid sequence comprises one or more polymorphisms associated with said allele.

The nucleic acid sample of the individual can be brought in contact with the immobilized polynucleotide or polynucleotides under conditions that ensure stringent hybridization, as discussed above. Hybridization of a sequence in the nucleic acid sample to an immobilized polynucleotide can be detected by any suitable method known in the art, including, but not limited to the methods discussed below.

In one embodiment of the invention, polynucleotide or polynucleotides can be used to detect the presence of a predisposing or protective allele by “reverse” dot blot hybridization. According to this method, a labeled polynucleotide can be immobilized on a membrane, as discussed above. The individual's nucleic acid sample can be added to the membrane. Then the labeled polynucleotide or a fragment thereof can be released from the membrane in such a way that a detection means can be used to determine if a sequence in the sample hybridized to the labeled nucleic acid molecule or molecules. This procedure, known as oligomer restriction, is described more fully in U.S. Pat. No. 4,683,194, which is incorporated herein by reference in its entirety. Alternatively, a polynucleotide immobilized to the membrane can bind or “capture” a part, or the whole allele from the nucleic acid sample and this “captured” nucleic acid can be detected by a second labeled nucleic acid molecule. Examples of methods to detect a labeled nucleic acid molecule or polynucleotide are discussed above, in Section 5.3.5.1.1.

5.3.5.2 Detecting the Amplicon of a Polymerase Chain Reaction

In some embodiments, the presence of predisposing or protective alleles can be detected by detecting the presence of an amplicon in amplification reactions. In a preferred embodiment, the amplification reaction can be PCR. According to the method of this embodiment, the individual's nucleic acid sample can be amplified and the amplicon detected by any amplification and detection method known in the art, including, but not limited to, methods described in U.S. Pat. Nos. 6,197,563; 6,171,785; 6,040,166; 5,773,258; 5,677,152; 5,665,548 and PCT Publication No. WO 89/04875, each of which is incorporated herein by reference.

In one embodiment, the amplification primers can hybridize under, for example, stringent hybridization conditions to a sequence on an allele in the nucleic acid sample that is being amplified, wherein the sequence comprises one or more polymorphisms associated with the allele. Stringent hybridization conditions are known in the art, and are described, for example, in Sambrook et al., supra. The amplification of the type 1 diabetes-associated allele can be used as confirmation of the presence of the particular allele.

In another embodiment, the primer need not hybridize to a polymorphism-comprising sequence on an allele. In this embodiment, the primer could bind to a region upstream (or 5′) of the polymorphism such that the sequence comprising the polymorphism is amplified. The presence of the type 1 diabetes-associated allele could be detected by a second polynucleotide which is specific for sequences in the type 1 diabetes-associated allele by methods such as, but not limited to, those described in Section 5.3.5.1 above.

5.3.5.3 Sequencing of the Individual's DNA or RNA

The presence of a predisposing or protective allele can also be detected by sequencing the nucleic acid sample collected from the individual or, by sequencing the amplified nucleic acid from the sample by any method known in the art. For example, the DNA obtained from the individual can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. See also, the techniques described in Sambrook et al., supra, and Ausubel et al., supra.

5.3.6 Assessing an Individual's Risk

Once the presence or absence of one or more type 1 diabetes-associated alleles have been detected in an individual, the individual's risk for the disease can be assessed based on the alleles detected. The presence of a predisposing allele can indicate that the individual has an increased risk for type 1 diabetes and therefore can have a greater likelihood of getting type 1 diabetes than an individual without the allele. On the other hand, the presence of a protective allele can correlate to a decreased risk for type 1 diabetes and the individual can have a lower likelihood of getting type 1 diabetes than an individual without the allele. When both a predisposing and a protective allele are present in an individual, then the effect of the predisposing allele can be partially decreased by the protective allele and vice versa.

The overall risk of the individual can be determined based on the type 1 diabetes-associated alleles present, the population of the individual and family history according to methods known to those of skill in the art.

This invention can, therefore, also be used to HLA type a panel in the class I or class II HLA loci and determine an individual's overall risk to any autoimmune diseases, for example, type 1 diabetes.

5.4 Reagents for Detecting Increased Risk for Autoimmune Diseases

The present invention also provides a reagent useful for detecting whether an individual has an increased risk for an autoimmune disease. In a preferred embodiment, the autoimmune disease is type 1 diabetes. Examples of reagents provided by the invention include, but are not limited to, one or more polynucleotides that hybridize under stringent hybridization conditions to one or more polymorphisms associated with a predisposing allele, one or more reagents used to amplify the individual's nucleic acid and detect the presence of a predisposing allele, and one or more reagents used to sequence the individual's nucleic acid thereby detecting the presence of a predisposing allele.

In one embodiment, the reagent for detecting whether an individual has an increased risk for an autoimmune disease such as type 1 diabetes can comprise one or more polynucleotides that hybridize under stringent hybridization conditions to one or more polymorphisms associated with a predisposing allele. The polynucleotide or polynucleotides can thus be used to identify one or more type 1 diabetes-associated alleles. In some embodiments, the polynucleotide or polynucleotides can hybridize to a nucleic acid sequence of a predisposing allele, wherein the nucleic acid sequence comprises one or more polymorphisms associated with the predisposing allele. The polynucleotide or polynucleotides can be designed or selected by techniques known to those of skill in the art. Additionally, hybridization conditions such as temperature, pH, nucleic acid length, nucleic acid sequence etc. is also within the knowledge of those of skill in the art. See Sambrook et al., supra, and Ausubel et al., supra, each of which is incorporated herein in its entirety. Section 6.1, Example 1, provides hybridization and wash conditions that can be used with the polynucleotides of the invention. Examples of sequences of polynucleotides that can be used with the invention for detecting whether an individual has an increased risk for an autoimmune disease such as type 1 diabetes include, but are not limited to, those listed in Tables 7 and 8.

In a preferred embodiment, the polynucleotide comprises a polynucleotide sequence that is fully complementary to a nucleic acid sequence in a predisposing allele, wherein the nucleic acid sequence comprises one or more polymorphisms associated with the predisposing allele. In certain embodiments, the polynucleotide comprises a polynucleotide sequence that is fully complementary to a nucleic acid sequence in a predisposing class I HLA allele. Preferably, the polynucleotide comprises a polynucleotide sequence that is fully complementary to a nucleic acid sequence in a predisposing HLA-C allele. More preferably, the polynucleotide comprises a polynucleotide sequence that is fully complementary to a nucleic acid sequence in the second or third exon of a predisposing HLA-C allele. Examples of predisposing alleles include, but are not limited to, HLA-C*0102, HLA-C*0302 and those described supra.

In some embodiments, the reagent for detecting whether an individual has an increased risk for an autoimmune disease such as type 1 diabetes includes one or more polynucleotides that can hybridize under stringent hybridization conditions to HLA-C*0102. Examples of such nucleic acid molecules include, but are not limited to, those that comprise a polynucleotide sequence selected from the group consisting of: SEQ. ID. NO: 5, SEQ. ID. NO: 6, SEQ. ID. NO: 7, SEQ. ID. NO: 8, SEQ. ID. NO: 9, SEQ. ID. NO: 10, SEQ. ID. NO: 11, SEQ. ID. NO: 12, SEQ. ID. NO: 13 and polynucleotide sequences complementary thereto (Table 7). In certain embodiments, multiple reagents that comprise combinations of 2, 3, 4, 5, 6, 7, 8, or 9 of the above sequences can be used to detect the presence of HLA-C*0102.

In some embodiments, the reagent for detecting whether an individual has an increased risk for an autoimmune disease such as type 1 diabetes includes one or more polynucleotides that can hybridize under stringent hybridization conditions to HLA-C*0302. Examples of such polynucleotides include, but are not limited to, those that comprise a polynucleotide sequence selected from the group consisting of: SEQ. ID. NO: 6, SEQ. ID. NO: 7, SEQ. ID. NO: 8, SEQ. ID. NO: 9, SEQ. ID. NO: 13, SEQ. ID. NO: 14, SEQ. ID. NO: 15, SEQ. ID. NO: 16, SEQ. ID. NO: 17 and polynucleotide sequences complementary thereto (Table 8). In certain embodiments, multiple reagents that comprise combinations of 2, 3, 4, 5, 6, 7, 8, or 9 of the above polynucleotides can be used to detect the presence of HLA-C*0302.

In certain embodiments, a particular class I HLA locus can conveniently be distinguished from other HLA loci by characteristic sequences of the class I HLA locus. For example, in one embodiment, sequences from exon 2 or exon 3 of class I HLA-C locus can be used to distinguish the HLA-C locus from other HLA loci and thereby to identify the HLA-C locus. Examples of sequences from exon 2 and exon 3 of class I HLA-C locus include, but are not limited to, SEQ. ID. NO: 1 and SEQ. ID. NO: 2, depicted below, respectively.

| | |
|---|---|
| SEQ. ID. NO: 1: | XCCGGAGTATTGGGACCGGGAGA |
| SEQ. ID. NO: 2: | XGCCTACGACGKCAAGGATTACATC |

5.5 Reagents for Detecting Decreased Risk for Autoimmune Diseases

The present invention also provides reagents useful for detecting whether an individual has a decreased risk for an autoimmune disease. In a preferred embodiment, the autoimmune disease is type 1 diabetes. Examples of the reagents include, but are not limited to, one or more polynucleotides that hybridize under stringent hybridization conditions to one or more polymorphisms associated with a protective allele, one or more reagents used to amplify the individual's nucleic acid and detect the presence of a protective allele, and one or more reagents used to sequence the individual's nucleic acid thereby detecting the presence of a protective allele.

In one embodiment, the reagent for detecting whether an individual has a decreased risk for an autoimmune disease such as type 1 diabetes can comprise one or more polynucleotides that hybridize under stringent hybridization conditions to one or more polymorphisms associated with a protective allele. The polynucleotide or polynucleotides can thus be used to identify one or more type 1 diabetes-associated alleles. In some embodiments, a polynucleotide can hybridize to a nucleic acid sequence of a protective allele, wherein the nucleic acid sequence comprises one or more polymorphisms associated with the protective allele. The polynucleotide can be designed or selected by techniques known to those of skill in the art. Additionally, hybridization conditions such as temperature, pH, nucleic acid length, nucleic acid sequence etc. is also within the knowledge of those of skill in the art. See Sambrook et al., supra, and Ausubel et al., supra. Section 6.1, Example 1, provides hybridization and wash conditions that can be used with the polynucleotides of the invention. Examples of sequences of nucleic acid molecules that can be used with the invention for detecting whether an individual has a decreased risk for an autoimmune disease such as type 1 diabetes include, but are not limited to, those listed in Tables 9-11.

In a preferred embodiment, the polynucleotide comprises a polynucleotide sequence that is fully complementary to a nucleic acid sequence in a protective allele, wherein the nucleic acid sequence comprises one or more polymorphisms associated with the protective allele. In a preferred embodiment, the polynucleotide comprises a polynucleotide sequence that is fully complementary to a nucleic acid sequence in a protective class I HLA allele. Preferably, the polynucleotide comprises a polynucleotide sequence that is fully complementary to a nucleic acid sequence in a protective HLA-A or HLA-C allele. More preferably, the polynucleotide comprises a polynucleotide sequence that is fully complementary to a nucleic acid sequence in the second or third exon of a protective HLA-A or HLA-C allele. Examples of protective alleles include, but are not limited to HLA-A*101, HLA-C*0702, HLA-C*1502 and those described supra.

In one embodiment, the reagent for detecting whether an individual has a decreased risk for an autoimmune disease such as type 1 diabetes includes one or more polynucleotides that can hybridize under stringent hybridization conditions to HLA-A*1101, HLA-C*0702 or HLA-C*1502.

In some embodiments, the polynucleotide or polynucleotides can hybridize under stringent hybridization conditions to HLA-A*1101. Examples of such polynucleotides include, but are not limited to, those that comprise a polynucleotide sequence selected from the group consisting of: SEQ. ID. NO: 20, SEQ. ID. NO: 21, SEQ. ID. NO: 22 SEQ. ID. NO: 23, SEQ. ID. NO: 24, SEQ. ID. NO: 25, SEQ. ID. NO: 26, SEQ. ID. NO: 27, SEQ. ID. NO: 28, SEQ. ID. NO: 29, SEQ. ID. NO: 30 and polynucleotide sequences complementary thereto (Table 9). In certain embodiments, multiple reagents that comprise combinations of 2, 3, 4, 5, 6, 7, 8, 9, 10 or 11 of the above polynucleotides can be used to detect the presence of HLA-A*1101.

In some embodiments, the polynucleotide or polynucleotides can hybridize under stringent hybridization conditions to HLA-C*0702. Examples of such polynucleotides include, but are not limited to, those that comprise a polynucleotide sequence selected from the group consisting of: SEQ. ID. NO: 6, SEQ. ID. NO: 7, SEQ. ID. NO: 9, SEQ. ID. NO: 12, SEQ. ID. NO: 13, SEQ. ID. NO: 16, SEQ. ID. NO: 17, SEQ. ID. NO: 18, SEQ. ID. NO: 19, SEQ. ID. NO: 20 and polynucleotide sequences complementary thereto (Table 10). In certain embodiments, multiple reagents that comprise combinations of 2, 3, 4, 5, 6, 7, 8, 9 or 10 of the above polynucleotides can be used to detect the presence of HLA-C*0702.

In some embodiments, the polynucleotide or polynucleotides can hybridize under stringent hybridization conditions to HLA-C*1502. Examples of such polynucleotides include, but are not limited to, those that comprise a polynucleotide sequence selected from the group consisting of: SEQ. ID. NO: 7, SEQ. ID. NO: 8, SEQ. ID. NO: 12, SEQ. ID. NO: 13, SEQ. ID. NO: 14, SEQ. ID. NO: 15, SEQ. ID. NO: 17, SEQ. ID. NO: 21, SEQ. ID. NO: 22, SEQ. ID. NO: 23 and polynucleotide sequences complementary thereto (Table 11). In certain embodiments, multiple reagents that comprise combinations of 2, 3, 4, 5, 6, 7, 8, 9 or 10 of the above polynucleotides can be used to detect the presence of HLA-C*1502.

In certain embodiments, a particular class I HLA locus can conveniently be distinguished from other HLA loci by characteristic sequences of the class I HLA locus. For example, in one embodiment, sequences from exon 2 or exon 3 of class I HLA-A or HLA-C loci can be used to identify the HLA-A or -C loci, respectively. Examples of sequences from exon 2 and exon 3 of the HLA-C locus include, but are not limited to, SEQ. ID. NO: 1 and SEQ. ID. NO: 2, respectively. Examples of sequences from exon 2 or exon 3 of the HLA-A locus include, but are not limited to, SEQ. ID. NO: 3 and SEQ. ID. NO: 4, depicted below, respectively.

| | |
|---|---|
| SEQ. ID. NO: 3: | XGAGCCGCGGGCGCCGTGGATAGAGCAGGAG |
| SEQ. ID. NO: 4: | XGAGGACCTGCGCTCTTGGACCGCGGCGGAC |

5.6 Kits for Detecting the Presence of a Predisposing or Protective Allele

The present invention also provides a kit useful for detecting an increased or decreased risk for an autoimmune disease. In a preferred embodiment, the autoimmune disease is type 1 diabetes. The kit can comprise one or more polynucleotides capable of detecting type 1 diabetes-associated alleles as described herein, as well as instructions for its use to detect an increased or decreased risk for an autoimmune disease such as type 1 diabetes. In some embodiments, the polynucleotide comprises a polynucleotide sequence that is fully complementary to a nucleic acid sequence in a type 1 diabetes-associated class I HLA allele, wherein said nucleic acid sequence comprises one or more polymorphisms associated with said allele. In some embodiments, the polynucleotide can be used to detect the presence of a type 1 diabetes-associated class I HLA allele by hybridizing to the allele under stringent hybridizing conditions. In some embodiments, the polynucleotide can be used as an extension primer in either an amplification reaction such as PCR or a sequencing reaction, wherein the type 1 diabetes-associated class I HLA allele is detected either by amplification or sequencing, respectively, as discussed above. In some embodiments, the kit can comprise one or more polynucleotides for detecting the presence of more than one type 1 diabetes-associated class I HLA allele. In some embodiments, the kit can comprise one or more polynucleotides for detecting the presence of combinations of predisposing alleles, protective alleles or both.

Further, the kit can comprise additional polynucleotides, e.g., sequencing or amplification primers or both which can, but need not, be sequence-specific to an type 1 diabetes-associated allele. The kit can further comprise one or more reagents useful for labeling a polynucleotide or an isolated nucleic acid molecule, e.g., one or more labeled or unlabeled NTPs or dNTPs (e.g., a mixture of dATP, dGTP, dCTP, dTTP and/or dUTP), one or more enzymes (e.g., DNA polymerase, kinase), one or more labeled or unlabeled primers etc. In some embodiments, the kit can additionally include one or more reagents useful for detecting a labeled moiety. Examples of such reagents include, but are not limited to, those discussed supra.

In some embodiments, the kit can additionally include one or more reagents useful for amplifying a nucleic acid of interest, including but not limited to, one or more amplification primers, one or more nucleotide triphosphates (“NTPs”) or deoxynucleotide triphosphates (“dNTPs”) (e.g., a mixture of dATP, dGTP, dCTP, dTTP and/or dUTP) one or more polymerizing enzymes etc.

In some embodiments, the kit can include one or more additional reagents useful for sequencing a nucleic acid of interest, e.g., one or more sequencing primers (labeled or unlabeled), one or more NTPs or dNTPs (e.g., a mixture of dATP, dGTP, dCTP, dTTP and/or dUTP), one or more labeled or unlabeled terminators (e.g., ddATP, ddGTP, ddCTP, ddTTP and/or ddUTP), one or more polymerizing enzymes (e.g., DNA polymerase) etc.

5.7 Arrays or Chips for Detecting the Presence of a Predisposing and/or Protective Allele

The present invention also provides an array or a chip useful for detecting an increased or decreased risk for an autoimmune. In a preferred embodiment, the autoimmune disease is type 1 diabetes. The array or chip can comprise one or more polynucleotides capable of detecting type 1 diabetes-associated alleles as described herein. In one embodiment, a predisposing or protective allele can be identified using an array of polynucleotides of the invention immobilized to a substrate or a “gene chip” (see, e.g. Cronin, et al., 1996, Human Mutation 7:244-255).

An array can provide a medium for matching known and unknown nucleic acid molecules based on base-pairing rules and automating the process of identifying the unknowns. An array experiment can make use of common assay systems such as microplates or standard blotting membranes, and can be worked manually, or make use of robotics to deposit the sample. The array can be a macro-array or a micro-array. The difference between a macro- and a micro-array generally is the size of the nucleic acid spots. Typically, a macro-array can contain spot sizes of about 300 microns or larger and can be easily imaged by existing gel and blot scanners. The sample spot sizes in a micro-array are typically less than 200 microns in diameter and a micro-array can comprise thousands of spots. The spot sizes can be designed or selected by those of skill in the art. Additionally, a micro-array may require additional specialized robotics and imaging equipment or specialized handling, which would be within the knowledge of those of skill in the art.

In some embodiments, arrays or chips, for example, DNA arrays, or DNA (gene) chips can be fabricated by high-speed robotics on a solid support or substrate, e.g., glass or nylon substrates. Polynucleotides of the invention, with known sequence identity, that can hybridize under stringent hybridization conditions to one or more polymorphisms associated with a type 1 diabetes-associated class I HLA allele can be immobilized on the substrate. The array or chip can then be contacted with a nucleic acid sample obtained from an individual whose risk for type 1 diabetes is being tested under stringent hybridization conditions. The pattern of hybridization detected on the array or chip is indicative of the alleles present in the individual's nucleic acid sample. Thus, arrays or chips can be used to detect the presence of one or more predisposing alleles or protective alleles or both. The nucleic acid molecule or molecules to be immobilized on the substrate can be designed or selected by techniques known to those of skill in the art. Additionally, hybridization conditions such as temperature, pH, etc. is also within the knowledge of those of skill in the art.

In some embodiments, the present invention provides an array for determining an individual's risk for type 1 diabetes comprising one or more polynucleotides immobilized on a substrate, wherein each polynucleotide individually comprises a sequence that hybridizes under stringent hybridization conditions to a nucleic acid sequence in a type 1 diabetes-associated class I HLA allele, wherein said nucleic acid sequence comprises one or more polymorphisms associated with said allele. In some embodiments, each polynucleotide individually comprises a sequence that is fully complementary to a nucleic acid sequence in a type 1 diabetes-associated class I HLA allele, wherein said nucleic acid sequence comprises one or more polymorphisms associated with said allele. The nucleic acid molecule or molecules immobilized on the substrate can, but need not, be labeled as discussed infra.

In embodiments, the immobilized polynucleotide or polynucleotides can each be individually complementary to a nucleic acid sequence in a predisposing class I HLA-C allele, preferably to a nucleic acid sequence in exon 2 or exon 3 of a predisposing class I HLA-C allele. In some embodiments, the alleles are HLA-C*0102 or HLA-C*0302. Examples of polynucleotides that can be immobilized on a substrate include, but are not limited to, those that comprise a polynucleotide sequence selected from the group consisting of: SEQ. ID. NO: 5, SEQ. ID. NO: 6, SEQ. ID. NO: 7, SEQ. ID. NO: 8, SEQ. ID. NO: 9, SEQ. ID. NO: 10, SEQ. ID. NO: 11, SEQ. ID. NO: 12, SEQ. ID. NO: 13 and polynucleotide sequences complementary thereto (Table 7) or those that comprise a polynucleotide sequence selected from the group consisting of: SEQ. ID. NO: 6, SEQ. ID. NO: 7, SEQ. ID. NO: 8, SEQ. ID. NO: 9, SEQ. ID. NO: 13, SEQ. ID. NO: 14, SEQ. ID. NO: 15, SEQ. ID. NO: 16, SEQ. ID. NO: 17 and polynucleotide sequences complementary thereto (Table 8). In certain embodiments, multiple polynucleotides that comprise combinations of 2, 3, 4, 5, 6, 7, 8, or 9 of the above groups of sequences can be immobilized on the substrate.

In preferred embodiments, the immobilized polynucleotide or polynucleotides can each be individually complementary to a nucleic acid sequence in a protective class I HLA-C allele, preferably to a nucleic acid sequence in exon 2 or exon 3 of a protective class I HLA-C allele. In some embodiments, the alleles are HLA-C*0702 or HLA-C*1502. Examples of polynucleotides that can be immobilized on a substrate include, but are not limited to, those that comprise a polynucleotide sequence selected from the group consisting of: SEQ. ID. NO: 6, SEQ. ID. NO: 7, SEQ. ID. NO: 9, SEQ. ID. NO: 12, SEQ. ID. NO: 13, SEQ. ID. NO: 16, SEQ. ID. NO: 17, SEQ. ID. NO: 18, SEQ. ID. NO: 19, SEQ. ID. NO: 20 and polynucleotide sequences complementary thereto (Table 10) or those that comprise a polynucleotide sequence selected from the group consisting of: SEQ. ID. NO: 7, SEQ. ID. NO: 8, SEQ. ID. NO: 12, SEQ. ID. NO: 13, SEQ. ID. NO: 14, SEQ. ID. NO: 15, SEQ. ID. NO: 17, SEQ. ID. NO: 21, SEQ. ID. NO: 22, SEQ. ID. NO: 23 and polynucleotide sequences complementary thereto (Table 11). In certain embodiments, multiple polynucleotides that comprise combinations of 2, 3, 4, 5, 6, 7, 8, 9 or 10 of the above groups of sequences can be immobilized on the substrate.

In preferred embodiments, the immobilized polynucleotide or polynucleotides can each be individually complementary to a nucleic acid sequence in a protective class I HLA-A allele, preferably to a nucleic acid sequence in exon 2 or exon 3 of a protective class I HLA-A allele. In some embodiments, the allele is HLA-A*1101. Examples of polynucleotides that can be immobilized on a substrate include, but are not limited to, those that comprise a polynucleotide sequence selected from the group consisting of: SEQ. ID. NO: 20, SEQ. ID. NO: 21, SEQ. ID. NO: 22 SEQ. ID. NO: 23, SEQ. ID. NO: 24, SEQ. ID. NO: 25, SEQ. ID. NO: 26, SEQ. ID. NO: 27, SEQ. ID. NO: 28, SEQ. ID. NO: 29, SEQ. ID. NO: 30 and polynucleotide sequences complementary thereto (Table 9). In certain embodiments, multiple polynucleotides that comprise combinations of 2, 3, 4, 5, 6, 7, 8, 9, 10 or 11 of the above groups of sequences can be immobilized on the substrate.

In some embodiments, the array can be used to detect the presence of one or more predisposing or protective HLA-C alleles. In preferred embodiments, the array can be used to detect the presence of combinations of two or more predisposing alleles, protective alleles or both.

The invention having been described, the following examples are intended to illustrate, and not limit, this invention.

### 6. EXAMPLES

As used in this section, “patients” refers to individuals with the disease, namely individuals with type 1 diabetes and “controls” refers normal individuals, those without the disease.

### 6.1 Example 1

### Identifying Predisposing Alleles and Protective Alleles

This example illustrates a method of identifying alleles which are associated with type 1 diabetes and characterizing them as potentially predisposing or protective.

The general approach was to use locus-specific primers to amplify the polymorphic segment of the HLA locus (exons 2 and 3 for class I loci) using biotinylated primers. The amplified product was then denatured and hybridized to an immobilized probe array under stringent (sequence-specific hybridization) conditions (see below). The hybridization of the labeled amplified product to a specific probe was then detected using a streptavidin-HRP conjugate and a soluble colorless substrate which was converted, in the presence of H₂O₂, into a blue precipitate. The immobilized probe array was made using SSO (“sequence-specific oligonucleotide”) probes, synthesized as BSA-oligonucleotides, and immobilized on a nylon membrane. The probe reactivity pattern was interpreted by a genotyping program. In some cases, a given probe reactivity pattern was consistent with more than a unique pair of alleles (“ambiguity”). In such cases, the ambiguity was generally resolved by amplifying the two alleles separately with group-specific primers and typing the PCR products. In other cases, likelihood considerations, based on allele frequencies and linkage disequilibrium patterns, was used to assign a unique genotype.

For purposes of this example, a Filipino population was chosen. Next, a DNA sample was extracted from patients and controls. Individuals were then HLA typed. Comparison of the alleles seen within the patient group with those seen in the control group provided a starting point for determining the role of the individual alleles.

Ninety patients (n=90) were selected for this study from amongst the Filipino population. The patients included in the study were affected by type 1 diabetes as defined by the recent ADA classification (The Expert Committee on the Diagnosis and Classification of Diabetes Mellitus 1997). The patients were born in the Philippines and all had two Filipino parents. These patients had been characterized for C-peptide levels below 0.3 mmol/l and for autoantibodies to islet cell autoantigens. Medici et al., 1999, Diabetes Care 22:1458. Samples were also collected from ninety-four Filipino normal subjects without a family history for diabetes. This was the control group. All patients and controls were from the southern region of Luzon, Philippines. The study was approved by the local Ethics Committee and informed consent was given by patients.

DNA was extracted and purified from 200 μl of frozen blood from patients and controls using QIA Amp blood kits. Genomic DNA was PCR amplified, and typed for HLA Class I (A, B, and C) loci. The HLA-A, B and C high resolution typing were carried out by co-amplification of exon 2 and 3 of each locus in a single PCR reaction using locus specific biotinylated primers and the amplicon was hybridized on a strip containing the immobilized sequence-specific oligonucleotide probes (“SSOP”).

Between 50-150 ng of genomic DNA were amplified in a 100 μl PCR reaction containing 50 mM KCl, 10 mM Tris-HCl (pH 8.3), 1.5 mM MgCl₂, 200 μM each of dATP, dCTP, dGTP, 400 μM dUTP, 0.50 μM each of biotinylated amplification primer, 1.0 unit of Taq DNA polymerase, and 15% glycerol. The amplification was carried out using a Perkin-Elmer DNA Thermal Cycler (Perkin-Elmer GeneAmp PCR System 9600, Perkin-Elmer Instruments, Foster City, Calif.) using a three-step temperature cycle:

(1) 15 s denaturation at 95° C.

(2) 45 s annealing at 60° C.

(3) 15 s extension at 72° C.

The PCR products were run and visualized using gel electrophoresis on a 4% Nusieve mix with 1% Seakem Agarose gel stained with ethidium bromide.

After the PCR amplification process, the amplicons were chemically denatured to form single strands which were then added to a well of a typing tray that contained a nylon membrane with bound, sequence-specific, oligonucleotide probes. The biotin-labeled amplicons bound (hybridized) to the sequence-specific probes and thus were “captured” onto the membrane strip. The stringent conditions for hybridization of the amplicons to the probes ensured the specificity of the reaction.

Seventy μl of denatured PCR product were hybridized to nylon membrane strips containing the immobilized SSOPs for 30 min at 50° C. in 4×SSPE (sodium phosphate solution with NaCl and EDTA)/0.5% SDS (sodium dodecyl sulfate). The strips were then rinsed briefly (a few seconds) in 1×SSPE/0.1% SDS at ambient temperature (25° C.), followed by a stringent wash in 1×SSPE/0.1% SDS for 15 min at 50° C. Immediately following the stringent wash, the strips were shaken in conjugate solution containing 5 ml 1×SSPE/0.1% SDS and 15 μl of SA-HRP (streptavidin-horseradish peroxidase enzyme conjugate) for 15 min on an orbital shaker at room temperature. The unbound SA-HRP was removed with two washes in 1×SSPE/0.1% SDS, 5 min each wash, followed by a rinse in 100 mM citrate buffer for 5 min. Color development and detection of probe hybridization was achieved by adding 4.0 ml of a 100 mM citrate solution containing 0.01% H₂O₂ mixed with 1.0 ml of 0.1% 3,3′,5,5′-tetramethylbenzidine (TMB) in 40% dimethyl formamide (4.0 ml Substrate A mixed with 1.0 ml Substrate B, Dynal, Inc., Lake Success, N.Y.) for 10 min and then the reaction was immediately stopped with three 5 min distilled water washes and the strips were photographed for genotype analysis. The reactions were carried out in a DYNAL AutoRELI SSO 48-well typing tray fitted for the DYNAL AutoRELI™ automated hybridization and detection instrument.

The typing and analysis was carried out by a computer program based on the SSOP hybridization pattern. The computer program used for the typing and analysis allows interpretation of the probe binding pattern and assigns the sample genotype. The program also warns the user of possible contamination if, in any given region more than two probes show up as positive signals. Alternatively, a strip scanner can be used to automate the genotype assignment. To facilitate throughput, two loci can be co-amplified and, because 83 probes can be immobilized on a single nylon membrane strip, in some cases, two loci can be typed with a single PCR and hybridization.

Comparisons between patients and controls were carried out with 2 by k tests for heterogeneity, using the log likelihood ratio test or G statistic (see Sokal and Rohlf, 1995, Biometry W.H. Freeman, San Francisco), where k is the number of allele, haplotype or genotype categories under consideration. Results for the overall heterogeneity having k−1 degrees of freedom are presented along with the G test statistic for each tested category (see Tables 1-3 below). Categories having a total of fewer than three samples were combined for testing. In order to indicate the direction and magnitude of nominally significant (P<0.05) differences between patients and controls for a category, odds ratios were given. The statistic W was employed to estimate the overall effect size of HLA on type 1 diabetes. Medici et al., 1999, Diabetes Care 9:1458-62; Sokal and Rohlf, 1995, Biometry W.H. Freeman, San Francisco. W had a value of one when two distributions had no variable categories in common, and a value of zero when the two distributions had identical proportions. Sokal and Rohlf, 1995, Biometry W.H. Freeman, San Francisco; Cohen, 1988, Statistical Power Analysis for the Social Sciences, Lawrence Erlbaum Associates, Hillsdale, N.J.; Klitz et al., 1995, Am J Hum Genet 57:1436-1444. Haplotype frequencies were estimated from patient and control samples separately with an EM algorithm described by Long et al. (Long et al., 1995, Am. J. Hum. Genet. 56:779-810) using the program of Baur and Danilov (Baur and Danilov 1980, Histocompatibility Testing 1980, 17 UCLA Tissue Typing Laboratory, Los Angeles). The estimated haplotype frequencies were used to calculate linkage disequilibrium values. The statistic D′ was used as a measure of relative disequilibrium. Lewontin, 1964, Genetics 49:49-67.

Using the information on haplotypes in which two alleles are in positive disequilibrium, it is possible to consider explanations for type 1 diabetes associations with the class I HLA alleles due to LD with other predisposing alleles. Some of the observed disease associations can be attributed to LD with high risk haplotypes while others cannot.

Individual alleles analyzed for the role they play in type 1 diabetes and the odds ratios associated with them are listed in Tables 1-3. Methods used to characterize the alleles as either predisposing or protective are described in Examples 2-4.

### 6.2 Example 2

### Characterizing an HLA-A Protective Allele

This example demonstrates the characterization of an HLA-A allele, HLA-A*1101, as negatively associated with type 1 diabetes, i.e., characterizing HLA-A*1101 as a protective allele.

Ninety patients and ninety-four normal subjects (“controls”) were selected for this study from amongst a Filipino population as described in Example 1. DNA was extracted and purified from a sample of blood taken from the patients and controls and the HLA class I high resolution typing were carried out as described in Example 1.

The HLA-A allele frequencies among patients and controls are shown in Table 1 and Table 2. Of the 20 HLA-A alleles identified in this population (Table 1), 13 were common enough to be tested independently, with the remaining 7 rare alleles pooled into a single combined class for the overall test. The HLA-A*1101 allele appeared protective (0.156 vs. 0.261), with an odds ratio of 0.51 (P=0.010).

The allele A*2402 was individually predisposing with an odds ratio of 1.9 (P=0.027). The A*24 allele group has been reported to be increased among Caucasian patients (see Fennessy et al., 1994, Diabetologia 37:937-944) and associated with early onset of disease (Nakanishi et al., 1999, J. Clin. Endocrinol. Metab. 84:3721-3725; Tait et al., 1995, Hum. Immunol. 42:116-122; Demaine et al., 1995, Diabetologia 38:632-38), justifying statistical testing among the A*24 alleles as a discrete group (Table 2). Our studies indicated that, in the Human Biological Data Interchange (“HBDI”) families (European origin), A*2402, the only A24 allele present, was associated with disease as well as with early onset of disease. The allelic diversity present within the A*24 group among Filipinos permitted comparison of the disease associations of different A*24 subtypes. The A*2402 and A*2403 allele frequencies were increased among the patient group. However, the other four A*24 alleles, in particular A*2407, appeared to be decreased, making the various A*24 alleles statistically heterogeneous for type 1 diabetes susceptibility (Table 2). The odds ratio for the A*2402 and A*2403 alleles combined was significant (OR=1.85, P=0.008).

Two locus haplotypes in significant linkage disequilibrium for pairs of the three class I loci, A-C, A-B and B-C, and for each of the class I HLA loci with DRB1 in the control sample are reported in Table 4. Using the information on haplotypes in positive disequilibrium (Table 4), it is possible to consider explanations for type 1 diabetes associations with the HLA class I region due to linkage disequilibrium with high risk DRB1 alleles. Among Filipinos, the high risk DRB1 alleles strongly associated with type 1 diabetes were, DRB*0301, *0405 and *0901. Some of the observed single and two locus disease associations can be attributed to LD with high risk DR-DQ haplotypes while others cannot. HLA-A*1101 is negatively associated with type 1 diabetes; this association might in part reflect the strong LD between A*1101 and DRB1*0803-DQB1*0601, a protective haplotype. However, A*1101 is also in LD with a susceptible or predisposing DR-DQ haplotype, DRB1*0901-DQB1*0303 so that the negative association can not be wholly attributable to LD with the DR-DQ region. The increase of A*3303 among patients (not significant) is attributable to LD with DRB1*0301-DQB1*0201/2. As noted above, A*2407, unlike A*2402, is decreased among patients (either neutral or slightly protective). A*2407 is in weak LD with DRB1*1101 and *1202, alleles that appear neutral or weakly protective. The risk differences between A*2402 and 2407 may reflect either differences in LD with DR-DQ haplotypes or they may reflect functional differences in the sequences of these alleles.

Comparing the distribution of two locus haplotypes in both patients and controls can reveal potential associations with specific combinations of alleles and help assess the role of individual alleles in susceptibility or protection. The frequency of two locus haplotype frequencies was estimated among both patients and controls. Because there are many more possible haplotypes than alleles at each of two loci, the available power to detect association effects is necessarily reduced. This is reflected in the increased number of haplotypes tested in two-locus combinations. The results of such haplotype frequency tests are summarized in Table 5. The frequencies of the fifteen A-C haplotypes sufficiently common for independent testing were very different between patients and controls (P=7×10⁻⁴). Two haplotypes were individually predisposing and two were individually protective. Because the A*1101 allele is found in each group, this might imply that this allele itself is not likely to be responsible for the observed effects (but, see below). The two negatively associated A-C haplotypes, *1101-*0702 and *3401-*1502, each contain HLA-C alleles seen as significantly protective in the C locus test (see Example 3). The test of the 12 most common A-B haplotypes revealed significant heterogeneity among patients and controls with the two significantly deviant haplotypes containing HLA-A alleles (A*2402, predisposing and A*1101, protective) noted as significant in the single locus test.

The frequency distributions of the A-DRB1 haplotypes were also significantly different among patients and controls. Two DRB1*0301-bearing haplotypes were predisposing, as were two protective haplotypes bearing DRB1*1502. One of these latter carried A*1101 which was seen as protective in combination with other A-DRB1 haplotypes as well. The A*2402-DRB1*0301 haplotype appears to confer higher risk (P=0.09) than the A*3303-DRB1*0301 haplotype, suggesting that specific combinations of HLA-A and DRB1 alleles determine the extent of disease risk

As candidates for independent class I influence on type 1 diabetes predisposition, A*1101 and A*2402 haplotypes with and without the presence of pertinent DRB1 alleles were examined (stratification analysis) (Table 6). A*1101 is in significant linkage disequilibrium with DRB1*0901, a strongly diabetogenic or predisposing DRB1 allele. A*1101 haplotype frequencies in the presence and absence of DRB1*0901 show that A*1101 without DRB1*0901 is protective (OR=0.47), DRB1*0901 alone is predisposing (OR=6.87) and when both alleles are present the risk is intermediate (OR=1.65). This implies that two independent influences, one protective and the other predisposing, tend to cancel each other out.

The haplotype tests with DRB1*1502, a known protective allele, and A*1101 revealed (Table 5) a strong negative association with disease. When both DRB1*1502 and A*1101 are present in an individual, strong disease protection is conferred (Table 6). The A*1101 haplotypes without DRB1*1502 are slightly protective, albeit not significantly (Table 6). The odds ratio in this case, 0.63, is significantly greater than that when both A*1101 and DRB1*1502 are present (OR=0.19). The DRB1*1502 risk without A*1101 is intermediate. This evidence suggests that A*1101 and DRB1*1502 may interact to produce greater protection.

The relationship of the nominally predisposing A*2402 with DRB1 diabetogenic influence can be similarly examined. DRB1*1502 is in significant positive linkage disequilibrium with A*2402 (Table 4). Tests of the presence and absence of these two alleles individuals demonstrates that A*2402 is predisposing in the absence of DRB1*1502 (OR=2.28), that haplotypes with only DRB1*1502 are protective, and carry significantly different risks. The combined haplotype is intermediate in risk (OR=0.85). In the A-DRB1 haplotype frequency tests, the combination A*2402-DRB1*0301 was significantly predisposing (Table 5) and more predisposing than the common A*3303-DRB1*0301 haplotype. It can be seen from Table 6 that A*2402 is predisposing in the absence of the diabetogenic DRB1*0301 (OR=1.75), while DRB1*0301 alone is somewhat more diabetogenic. Interestingly, the combined haplotype is significantly more diabetogenic than A*2402 alone. This suggests possible interactive effects for predisposition operating between the HLA-DR and HLA-A region.

A*24, defined serologically, has been reported to be associated with disease as well as with age of onset (Fujisawa et al, 1995). A study of the HBDI families using DNA-based HLA typing also implicated A*2402 as a disease risk factor, not attributable to linkage disequilibrium with high-risk DR-DQ haplotypes, that is also associated with age of onset. A*2402 was the only allele observed within the A*24 group in the HBDI families. Among Filipinos, however, A*24 consists of several distinct alleles, which appear to be heterogeneous with respect to risk; A*2402 and A*2403 were increased among patients while A*2407 was decreased. The differences in risk between A*2402+A*2403 and A*2407 (P<0.05 with OR 2.4) could reflect functional sequence differences or different patterns of linkage disequilibrium, or, conceivably, type 1 error. The increase of A*2402 and A*2403 among patients is not attributable to linkage disequilibrium (Table 5). A*2407 is in weak linkage disequilibrium with DRB1*1502 but this observation may not account for the differences in association between this HLA-A allele and A*2402 and A*2403. It should be noted that A*2407 differs from both A*2402 and A*2403 by a His to Gln change at position 70. This non-conservative amino acid change at a residue which contributes to peptide binding pockets B and C may be responsible for functional differences between the A24 alleles.

In addition to an increased diversity of alleles within the A*24 allele group, the Filipino population has a distinctive pattern of LD (Table 4). Several extended haplotypes can be inferred from this analysis; the most common includes A*2402. The very common allele DRB1*1502 (f=0.43) is part of the extended haplotype, A*2402-C*0702-B*3802-DRB1*1502-DQA1*0102-DQB1*0502-DPB1*01011.

Convincing evidence for the independent influence of class I alleles in Filipino type 1 diabetes requires careful consideration of the confounding influence due to LD of nearby HLA loci, especially that due to the DR-DQ class II region. Two HLA-A alleles, A*1101 and A*2402, demonstrated nominally significant associations with type 1 diabetes (Table 1). The overall evidence for these two alleles was examined. This examination led to the conclusion that these were producing, respectively, protective and predisposing influences on type 1 diabetes not attributable to LD with the class II region. The frequency of A*1101 is quite high in Filipinos (0.261), but only a small fraction of this (f=0.027) is accounted for by significant positive linkage disequilibrium with the diabetogenic allele DRB1*0901. It was also noted that A*1101 had a protective effect in combination with the DRB1*1502 protective allele implying the action of two independent mechanisms conferring disease protection. A*1101 was strongly protective in this population consistent with that seen in the HBDI families. Overall, it was noted that the extent of disease risk was determined by the specific combinations of susceptible and protective alleles.

### 6.3 Example 3

### Characterizing HLA-C Protective Alleles

This example demonstrates the characterization of HLA-C alleles, HLA-C*0702 and HLA-C*1502, as negatively associated with type 1 diabetes, i.e., characterizing HLA-C*0702 and HLA-C*1502 as protective alleles.

The methods described in Example 1 were used to obtain the DNA of individuals in the patient and control groups, to HLA type the individuals and determine which alleles were disease associated.

The HLA-C allele frequencies among patients and controls is shown in Table 3. At the HLA-C locus, 15 alleles were tested individually and 11 rare alleles were assigned to the combined category. The overall test of heterogeneity between patient and control frequencies was highly significant at the HLA-C locus, with P=0.007. Individually, HLA-C*0702 and C*1502 appeared protective.

Two locus haplotypes in significant linkage disequilibrium for pairs of the three class I loci, A-C, A-B and B-C, and for each of the class I HLA loci with DRB1 in the control sample are reported in Table 4. Over half (57%) of total haplotypes from the tightly linked B-C loci are present in haplotypes in positive linkage disequilibrium, with no single haplotype reaching a frequency of 10%; C*0702 with a frequency of 33% in the control sample is in significant LD with several different B alleles. HLA-C haplotypes in positive LD with HLA-A and HLA-DRB1 each comprised over 40% of the total. The common alleles, C*0702 and DRB1*1502, were present on haplotypes sharing alleles A*2402 and B*3802. This suggests the presence of a rather frequent extended haplotype in this population: A*2402-C*0702-B*3802-DRB1*1502. Toward the centromere, this extended haplotype contains DQB1*0502-DPA1*02022-DPB1*0101.

Using the information on haplotypes in positive disequilibrium (Table 4), it is possible to consider explanations for type 1 diabetes associations with the HLA class I region due to linkage disequilibrium with high risk DRB1 alleles. Among Filipinos, the high risk DRB1 alleles strongly associated with type 1 diabetes were, DRB*0301, *0405 and *0901. Some of the observed single and two locus disease associations can be attributed to LD with high risk DR-DQ haplotypes while others cannot. The HLA-C locus is the only individual class I locus that showed significant overall allele frequency differences among patients and controls (Table 3). At the C locus, HLA-C*0702 and HLA-C*1502 were both negatively associated with disease. The HLA-C*0702 negative association may be attributed to LD with the protective DRB1*1502 allele but HLA-C*1502 is in LD with the susceptible DRB1*0405 and therefore, the negative association of HLA-C*1502 cannot be attributed simply to LD with a protective DR-DQ haplotype.

Comparing the distribution of two locus haplotypes in both patients and controls can reveal potential associations with specific combinations of alleles and help assess the role of individual alleles in susceptibility or protection. The frequency of two locus haplotype frequencies was estimated among both patients and controls. Because there are many more possible haplotypes than alleles at each of two loci, the available power to detect association effects is necessarily reduced. This is reflected in the increased number of haplotypes tested in two-locus combinations. The results of such haplotype frequency tests are summarized in Table 5. The frequencies of the fifteen A-C haplotypes sufficiently common for independent testing were very different between patients and controls (P=7×10⁻⁴). The two negatively associated A-C haplotypes, A*1101-C*0702 and A*3401-C*1502, each contain HLA-C alleles seen as significantly protective in the HLA-C locus test (Table 3). Frequencies of the 14 B-C haplotypes did not differ among patients and controls. The C-DR haplotypes strongly discriminate patients from controls with a highly significant P value (P=2×10⁻⁷), having three predisposing and four protective haplotypes. In nearly each case, the association of these seven haplotypes conform to the susceptibility patterns seen for the associated DRB1 alleles.

As a candidate for independent class I influence on type 1 diabetes predisposition, the HLA-C*1502 haplotype with and without the presence of pertinent DRB1 alleles was examined (stratification analysis) (Table 6). The HLA-C*1502 allele was protective when tested with other HLA-C alleles (Table 3), and it is in significant positive disequilibrium with the diabetogenic (predisposing) allele, DRB1*0405 (Table 4). Haplotypic presence and absence testing (Table 6) shows that HLA-C*1502 is protective on its own (OR=0.16), but also that the combined haplotype C*1502 and DRB1*0405 is intermediate in risk between C*1502 and DRB1*0405, and significantly less than the risk associated with DRB1*0405. This suggests that C*1502 protection may act to reduce the risk of DRB1*0405.

One method to determine whether an allele itself is responsible for a protective or predisposing effect is to examine whether a uniform effect is observed for all haplotypes bearing that allele at a second locus. If the effect of an allele is not uniform, then it is unlikely that the allele is by itself responsible for the observed disease association, although this does not exclude the possibility of more complicated interactions between alleles at different loci. In fact, this analysis can suggest specific combinations of alleles that determine the extent of risk. The HLA-C alleles all demonstrated uniform predispositional or protective effects when divided according to haplotype, although only the test involving the common allele HLA-C*0702 had significant statistical power to detect any possible differences.

### 6.4 Example 4

### Characterizing HLA-C Predisposing Alleles

This example demonstrates the characterization of HLA-C alleles, HLA-C*0102 and HLA-C*0302, as positively associated with type 1 diabetes, i.e., characterizing HLA-C*0102 and HLA-C*0302 as predisposing alleles.

The methods described in Example 1 were used to obtain the DNA of individuals in the patient and control groups, to HLA type the individuals and determine which alleles were disease associated.

The HLA-C allele frequencies among patients and controls is shown in Table 3. At the HLA-C locus, 15 alleles could be tested individually with 11 rare alleles assigned to the combined category. The overall test of heterogeneity between patient and control frequencies was highly significant at the HLA-C locus, with P=0.007. Individually, HLA-C*0102 and HLA-C*0302 appeared predisposing, i.e., they were positively associated with type 1 diabetes.

Using the information on haplotypes in positive disequilibrium (Table 4), it is possible to consider explanations for type 1 diabetes associations with the HLA class I region due to linkage disequilibrium with high risk DRB1 alleles. Among Filipinos, the high risk DRB1 alleles strongly associated with type 1 diabetes were, DRB*0301, *0405 and *0901. Some of the observed single and two locus disease associations can be attributed to LD with high risk DR-DQ haplotypes while others cannot. The HLA-C locus is the only individual class I locus that showed significant overall allele frequency differences among patients and controls (Table 3). At the C locus, HLA-C*0102 and HLA-C*0302 were both positively associated with disease. The HLA-C*0302 association may reflect LD with DRB1*0301 but, based on analysis of the LD patterns, the association of HLA-C*0102 with type 1 diabetes is not simply attributable to LD with high-risk DR-DQ haplotypes. Thus, HLA-C*0102 itself, or some allele at a nearby locus in strong LD, may confer risk to type 1 diabetes.

Comparing the distribution of two locus haplotypes in both patients and controls can reveal potential associations with specific combinations of alleles and help assess the role of individual alleles in susceptibility or protection. The frequency of two locus haplotype frequencies was estimated among both patients and controls. Because there are many more possible haplotypes than alleles at each of two loci, the available power to detect association effects is necessarily reduced. This is reflected in the increased number of haplotypes tested in two-locus combinations. The results of such haplotype frequency tests are summarized in Table 5. The C-DR haplotypes strongly discriminate patients from controls with a highly significant P value (P=2×10⁻⁷), having three predisposing and four protective haplotypes. In nearly each case, the association of these seven haplotypes conform to the susceptibility patterns seen for the associated DRB1 alleles.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below.

All references cited herein are hereby incorporated by reference in their entireties.

TABLE 1
HLA-A Allele Frequencies in Filipino Patients and Controls

| HLA-A Allele | IDDM % | Controls % | G^{†} | Odds Ratio |
|---|---|---|---|---|
| 0101 | 1.1 | 0.5 | 0.4 | |
| 0201 | 6.1 | 6.9 | 0.1 | |
| 0203 | 0.6 | 0.5 | | |
| 0206 | 2.8 | 1.6 | 0.6 | |
| 0207/15N* | 1.1 | 1.6 | 0.2 | |
| 0211 | 0.6 | 0 | | |
| 0302 | 0.6 | 0 | | |
| 1101 | 15.6 | 26.1 | 4.9 | 0.5 |
| 1102 | 1.1 | 0.5 | 0.4 | |
| 2402/09N* | 33.3 | 21.3 | 4.9 | 1.9 |
| 24032 | 2.8 | 1.1 | 1.5 | |
| 2405 | 0 | 0.5 | | |
| 2407 | 9.4 | 13.8 | 1.5 | |
| 2410 | 0.6 | 1.6 | 1.0 | |
| 2601 | 1.3 | 2.8 | 1.2 | |
| 2902 | 0.6 | 0.6 | | |
| 3201 | 0.6 | 0 | | |
| 3303 | 11.0 | 7.4 | 1.0 | |
| 3401 | 11.0 | 13.3 | 0.4 | |
| 6801 | 0.6 | 0 | | |
| Combined | 3.3 | 1.6 | 1.2 | |
| Sum | | | 19.1 | |
| | | | df = 13 | |
| | | | p = 0.12 | |
| | | | W = 0.23 | |

Our typing system does not distinguish A*0207 from *0215N and A*2402 from *2409N. However, A*0215N and *2409N are extremely rare; we presume most if not all of these alleles are *0207 and *2402.
^{†}G statistic (see Sokal and Rohlf, 1995, Biometry W. H. Freeman, San Francisco).

TABLE 2
Test of Heterogeneity Among A*24 Allele Frequencies in
Filipino Patients and Controls

| A*24 allele | Patient (n = 83) % | Control (72) % | G^{†} |
|---|---|---|---|
| *2402 | 75.9 | 55.6 | 1.7 |
| *2403 | 6.0 | 2.8 | 0.9 |
| *2405 | 0.0 | 1.4 | |
| *2407 | 20.5 | 36.1 | 3.4 |
| *2410 | 1.2 | 4.2 | |
| Combined rare | 1.2 | 5.6 | 2.4 |
| alleles | | | |
| Sum | | | 8.4 |
| | | | df = 3, P < 0.05 |

^{†}G statistic (see Sokal and Rohlf, 1995, Biometry W. H. Freeman, San Francisco).

TABLE 3
HLA-C Allele Frequencies in Filipino Patients and Controls

| HLA-C Allele | IDDM % | Controls % | G^{†} | Odds Ratio |
|---|---|---|---|---|
| 0102 | 8.9 | 3.7 | 4.1 | 2.6 |
| 02022 | 0.6 | 0 | | |
| 0302 | 12.2 | 6.4 | 3.6 | 2.1 |
| 0303 | 4.4 | 1.6 | 2.6 | |
| 03041 | 2.8 | 6.4 | 2.6 | |
| 0305 | 1.1 | 0 | | |
| 0401/05 | 10.6 | 12.2 | 0.2 | |
| 0402 | 0.6 | 0 | | |
| 0403 | 6.7 | 8.0 | 0.2 | |
| 0406 | 1.1 | 1.6 | 0.2 | |
| 0501/02 | 0.6 | 0 | | |
| 0602 | 2.8 | 1.1 | 0.8 | |
| 0701 | 2.2 | 1.1 | 2.3 | |
| 0706 | 1.1 | 0 | | |
| 0702 | 21.7 | 33.0 | 4.1 | 0.58 |
| 0704 | 1.7 | 2.1 | 0.1 | |
| 0801 | 13.9 | 10.6 | 0.9 | |
| 0802 | 0 | 0.5 | | |
| 1202 | 0.6 | 0.5 | | |
| 1203 | 0.6 | 0.5 | | |
| 12042 | 1.7 | 0.5 | 1.2 | |
| 1402 | 0.6 | 1.6 | 0.9 | |
| 1502 | 2.8 | 8.0 | 4.1 | 0.4 |
| 1601 | 0 | 0.5 | | |
| 1604 | 0.6 | 0 | | |
| 1701/02 | 0.6 | 0 | | |
| Combined | 5.0 | 2.1 | 2.3 | |
| Sum | | | 31.5 | |
| | | | df = 15 | |
| | | | P = 0.007 | |
| | | | W = 0.29 | |

^{†}G statistic (see Sokal and Rohlf, 1995, Biometry W. H. Freeman, San Francisco).

TABLE 4
Two-point HLA Class I and DRB1 Haplotypes in
Significant Positive Disequilibrium in the
Filipino Control Population (2N = 188)

| | | |
|---|---|---|
| Haplotype | D' (%)¹ | Freq (%) |

A-C

| | | |
|---|---|---|
| 0201-0403 | 25** | 2.2 |
| 0201-0801 | 23* | 2.1 |
| 1101-0702 | 26** | 13.5 |
| 2402-0702 | 20* | 9.8 |
| 2402-0704 | 55* | 1.4 |
| 2407-0401 | 50*** | 6.3 |
| 3303-0302 | 73*** | 4.8 |
| 3401-0403 | 30** | 3.1 |
| 3401-1502 | 43*** | 3.8 |
| | | 47.0 |

A-B

| | | |
|---|---|---|
| 0201-1521 | 41*** | 2.7 |
| 1101-1301 | 73** | 4.3 |
| 1101-1502 | 51** | 3.7 |
| 1101-1532 | 43*** | 2.1 |
| 2402-0705 | 68** | 1.6 |
| 2402-3802 | 23* | 5.0 |
| 2402-4801 | 46* | 2.1 |
| 2407-3505 | 71*** | 6.4 |
| 3303-5801 | 60*** | 4.0 |
| 3401-1521 | 28*** | 2.2 |
| 3401-4002 | 54*** | 4.8 |
| | | 38.9 |

B-C

| | | |
|---|---|---|
| 0705-0702 | 64* | 1.6 |
| 1301-0303 | 24* | 1.6 |
| 1502-0801 | 69*** | 4.3 |
| 1513-0801 | 89*** | 2.4 |
| 1521-0403 | 100*** | 5.9 |
| 1532-0702 | 100*** | 3.7 |
| 3505-0401 | 79*** | 7.0 |
| 3801-0702 | 100** | 2.1 |
| 3802-0702 | 68*** | 6.7 |
| 3901-0702 | 100* | 1.6 |
| 4001-0401 | 34*** | 2.7 |
| 4002-1502 | 77*** | 5.9 |
| 4601-0102 | 100*** | 3.3 |
| 4801-0801 | 52*** | 2.1 |
| 5801-0302 | 96** | 6.1 |
| | | 57.0 |

A-DRB1

| | | |
|---|---|---|
| 1101-0803 | 38* | 2.9 |
| 1101-0901 | 50* | 2.7 |
| 2402-1502 | 33** | 13.5 |
| 2407-1202 | 20* | 3.7 |
| 3303-0301 | 47*** | 2.7 |
| 3401-1502 | 44** | 9.3 |
| | | 31.9 |

C-DRB1

| | | |
|---|---|---|
| 0302-0301 | 57*** | 3.2 |
| 0303-0803 | 40*** | 2.4 |
| 0303-0901 | 32*** | 1.6 |
| 0401-0403 | 62*** | 2.2 |
| 0401-1202 | 23*** | 3.5 |
| 0702-1502 | 45*** | 23.0 |
| 0704-1502 | 100* | 2.1 |
| 0801-1101 | 32** | 2.3 |
| 1502-0405 | 61*** | 3.7 |
| | | 44.0 |

B-DRB1

| | | |
|---|---|---|
| 1301-0803 | 16* | 1.1 |
| 1502-1202 | 41*** | 2.8 |
| 3505-1202 | 41*** | 4.1 |
| 3801-1502 | 100* | 2.1 |
| 3802-1502 | 92*** | 12.1 |
| 4002-0405 | 57*** | 3.2 |
| 5801-0301 | 58** | 3.2 |
| | | 25.7 |

¹Positive D' values expressed as percent.
P values:
*0.05;
**0.01;
***0.001.
The statistic D' was used as a measure of relative disequilibrium. Lewontin, 1964, Genetics 49: 49-67.

TABLE 5
Summary of Tests of HLA Two-Locus Haplotypes
on Type I Diabetes in Filipinos

| | | | | | Predisposing | | | |
|---|---|---|---|---|---|---|---|---|
| Loci | W^{‡} | G^{†} | df^{a} | P | Haplotype | OR^{b} | Haplotype | OR^{b} |
| A-C | 0.30 | 38.9 | 15 | 7 × 10⁻⁴ | 1101-0102 | 8.7* | 1101-702 | 0.2*** |
| | | | | | 2402-0302 | 12.9** | 3401-1502 | 0.2* |
| A-B | 0.26 | 28.9 | 12 | 0.004 | 2402-5801 | 18.6*** | 1101-3802 | 0.1** |
| B-C | 0.18 | 11.4 | 14 | ns | | | | |
| B-DR | 0.31 | 18.6 | 9 | 0.029 | 5801-0301 | 4.2** | 1502-1202 | 0.2* |
| C-DR | 0.41 | 74.6 | 23 | 2 × 10⁻⁷ | 0102-0901 | 8.6** | 0303-0803 | 0.1* |
| | | | | | 0302-0301 | 3.8** | 0401-0403 | 0.1* |
| | | | | | 0401-0901 | 6.4* | 0702-1202 | 0.2* |
| | | | | | | | 0702-1502 | 0.4** |
| A-DRB1 | 0.25 | 52.3 | 12 | 5 × 10⁻⁷ | 2402-0301 | 22.1*** | 0201-1502 | 0.1*** |
| | | | | | 3303-0301 | 2.9* | 1101-1502 | 0.1*** |

^{a}The number of haplotypes common enough to be included in the overall test is equivalent to the degrees of freedom for the log likelihood ratio test.
^{b}Odds ratio with associated P values from 2 × 2 tables: * <0.05, ** <0.01, *** <0.001.
^{†}G statistic (see Sokal and Rohlf, 1995, Biometry W. H. Freeman, San Francisco).
^{‡}See Medici et al., 1999, Diabetes Care 9: 1458-62; Sokal and Rohlf, 1995, Biometry W. H. Freeman, San Francisco.

TABLE 6
Stratification Tests of the Influence of Specific DRB1
Alleles on the Risk Associated with A*1101, A*2402 and
C*1502 for type I Diabetes in Filipinos

| | Relationship to DR Effect | | | |
|---|---|---|---|---|
| | | Frequencies | | |
| Allele | Haplotype¹ | T1D | Control | OR (95% CI)² |
| A*1101 | A*1101-DRB1*0901 | | | |
| | + + | 0.044 | 0.027 | 1.65 (0.55-4.92) |
| | − + | 0.111 | 0.015 | 6.87 (2.12-22.15) |
| | + − | 0.111 | 0.233 | 0.47 (0.26-0.83) |
| | − − | 0.733 | 0.725 | reference³ |
| | A*1101-DRB1*1502 | | | |
| | + + | 0.025 | 0.107 | 0.19 (0.07-0.50) |
| | − + | 0.242 | 0.317 | 0.53 (0.33-0.86) |
| | + − | 0.131 | 0.143 | 0.63 (0.34-1.18) |
| | − − | 0.603 | 0.433 | reference |
| A*2402 | A*2402- | | | |
| | DRB1*1502 | | | |
| | + + | 0.131 | 0.135 | 0.85 (0.45-1.63) |
| | − + | 0.136 | 0.317 | 0.39 (0.22-0.67) |
| | + + | 0.203 | 0.078 | 2.28 (1.18-4.41) |
| | − − | 0.531 | 0.470 | reference |
| | A*2402- | | | |
| | DRB1*0301 | | | |
| | + + | 0.050 | 0 | High (3.19-—)⁴ |
| | − + | 0.111 | 0.053 | 2.76 (1.26-6.05) |
| | + − | 0.283 | 0.213 | 1.75 (1.08-2.86) |
| | − − | 0.556 | 0.734 | reference |
| C*1502 | C*1502-DRB1*0405 | | | |
| | + + | 0.022 | 0.037 | 0.64 (0.20-2.09) |
| | − + | 0.128 | 0.021 | 6.43 (2.27-18.17) |
| | + − | 0.016 | 0.037 | 0.16 (0-1.012) |
| | − − | 0.844 | 0.904 | reference |

¹The presence or absence of a particular allele is indicated with a plus or minus.
²The frequencies of haplotypes having one or both alleles are compared to the frequencies of haplotypes without either allele.
³Proportions of individual lacking either of the alleles were compared to each of the other three groups.
⁴The odds ratio and upper 95% CI is not defined when the control cell has no observations.

TABLE 7
Polynucleotides for the Detection of HLA-C*0102

| | |
|---|---|
| Name | Sequence |

| | |
|---|---|
| SEQ. ID. NO: 5 | XGACACGGATGTGAAGAAATAC |
| SEQ. ID. NO: 6 | XCTCCCCTCTCGGACTCGCG |
| SEQ. ID. NO: 7 | XGCCGCGGGCGCCGT |
| SEQ. ID. NO: 8 | XAGGCACAGACTGACCG |
| SEQ. ID. NO: 9 | XAGCCTGCGGAACCTGC |
| SEQ. ID. NO: 10 | XGGCGTACTGGTCATACCC |
| SEQ. ID. NO: 11 | XGCGGAGAGCCTACCTGG |
| SEQ. ID. NO: 12 | XGAGGGCACGTGCGTGG |
| SEQ. ID. NO: 13 | XCTCACCGGCCTCGCTCTG |

X = BSA

TABLE 8
Polynucleotides for the Detection of HLA-C*0302

| | |
|---|---|
| Name | Sequence |

| | |
|---|---|
| SEQ. ID. NO: 6 | XCTCCCCTCTCGGACTCGCG |
| SEQ. ID. NO: 7 | XGCCGCGGGCGCCGT |
| SEQ. ID. NO: 8 | XAGGCACAGACTGACCG |
| SEQ. ID. NO: 9 | XAGCCTGCGGAACCTGC |
| SEQ. ID. NO: 13 | XCTCACCGGCCTCGCTCTG |
| SEQ. ID. NO: 14 | XGGGACACAGCGGTGTAGAA |
| SEQ. ID. NO: 15 | XAGCCATACATCCTCTGGA |
| SEQ. ID. NO: 16 | XGTATGACCAGTCCGCCTA |
| SEQ. ID. NO: 17 | XGGAGCAGCTGAGAGCCTA |

X = BSA

TABLE 9
Polynucleotides for the Detection of HLA-A*1101

| | |
|---|---|
| Name | Sequence |

| | |
|---|---|
| SEQ. ID. NO: 24 | XATGAGGTATTTCTACACCTCCG |
| SEQ. ID. NO: 25 | XATTGGGACCAGGAGACAC |
| SEQ. ID. NO: 26 | XGGTCTGTGACTGGGCCTTCAT |
| SEQ. ID. NO: 27 | XCAGGTCCACTCGGTCAATCTGTGACT |
| SEQ. ID. NO: 28 | XCCATCCAGATAATGTATGGC |
| SEQ. ID. NO: 29 | XGGCGTCCTGCCGGTACC |
| SEQ. ID. NO: 30 | XGAACGAGGACCTGCGC |
| SEQ. ID. NO: 31 | XACTTGCGCTTGGTGATCT |
| SEQ. ID. NO: 32 | XGGCCCATGCGGCGGA |
| SEQ. ID. NO: 33 | XGAGCAGCAGAGAGCCTA |
| SEQ. ID. NO: 34 | XGAGGGCCGGTGCG |

X = BSA

TABLE 10
Polynucleotides for the Detection of HLA-C*0702

| | |
|---|---|
| Name | Sequence |

| | |
|---|---|
| SEQ. ID. NO: 6 | XCTCCCCTCTCGGACTCGCG |
| SEQ. ID. NO: 7 | XGCCGCGGGCGCCGT |
| SEQ. ID. NO: 9 | XAGCCTGCGGAACCTGC |
| SEQ. ID. NO: 12 | XGAGGGCACGTGCGTGG |
| SEQ. ID. NO: 13 | XCTCACCGGCCTCGCTCTG |
| SEQ. ID. NO: 16 | XGTATGACCAGTCCGCCTA |
| SEQ. ID. NO: 17 | XGGAGCAGCTGAGAGCCTA |
| SEQ. ID. NO: 18 | XACACGGCGGTGTCGAAATA |
| SEQ. ID. NO: 19 | XTCGGTCAGCCTGTGCCTG |
| SEQ. ID. NO: 20 | XAGAGGATGTCTGGCTGC |

X = BSA

TABLE 11
Polynucleotides for the Detection of HLA-C*1502

| | |
|---|---|
| Name | Sequence |

| | |
|---|---|
| SEQ. ID. NO: 7 | XGCCGCGGGCGCCGT |
| SEQ. ID. NO: 8 | XAGGCACAGACTGACCG |
| SEQ. ID. NO: 12 | XGAGGGCACGTGCGTGG |
| SEQ. ID. NO: 13 | XCTCACCGGCCTCGCTCTG |
| SEQ. ID. NO: 14 | XGGGACACAGCGGTGTAGAA |
| SEQ. ID. NO: 15 | XAGCCATACATCCTCTGGA |
| SEQ. ID. NO: 17 | XGGAGCAGCTGAGAGCCTA |
| SEQ. ID. NO: 21 | XGCGAGTCCAAGAGGGGAG |
| SEQ. ID. NO: 22 | XCGCAGTTTCCGCAGGTT |
| SEQ. ID. NO: 23 | XGTAGGCTAACTGGTCATGC |

X = BSA

What is claimed is:

1. A method for determining an individual's risk for type 1 diabetes comprising:
detecting the presence of a type 1 diabetes-associated class I HLA-C allele in a nucleic acid sample of the individual, wherein the presence of said allele indicates the individual's risk for type 1 diabetes.

2. The method of claim 1, wherein the individual is of Asian descent.

3. The method of claim 1, wherein the individual is of Filipino descent.

4. The method of claim 1, wherein the risk for type 1 diabetes is an increased risk.

5. The method of claim 4, wherein the allele is a disease-predisposing allele.

6. The method of claim 1, wherein the risk for type 1 diabetes is a decreased risk.

7. The method of claim 6, wherein the allele is a disease-protective allele.

8. The method of claim 1, wherein the nucleic acid sample comprises DNA.

9. The method of claim 1, wherein the nucleic acid sample comprises RNA.

10. The method of claim 1, wherein the nucleic acid sample is amplified.

11. The method of claim 10, wherein the nucleic acid sample is amplified by a polymerase chain reaction.

12. The method of claim 1, wherein the allele is detected by amplification.

13. The method of claim 12, wherein the allele is detected by a polymerase chain reaction.

14. The method of claim 1, wherein the allele is detected by sequencing.

15. The method of claim 1, wherein the allele is detected by contacting the nucleic acid sample with one or more polynucleotides that hybridize under stringent hybridization conditions to one or more polymorphisms associated with said allele and detecting hybridization.

16. The method of claim 15, wherein the one or more polynucleotides are each individually complementary to a sequence in exon 2 or exon 3 of a class I HLA-C allele.

17. The method of claim 15, wherein the one or more polynucleotides comprise at least one of the sequences listed in Table 7.

18. The method of claim 17, wherein the allele is HLA-C*0102.

19. The method of claim 15, wherein the one or more polynucleotides comprise at least one of the polynucleotide sequences listed in Table 11.

20. The method of claim 19, wherein the allele is HLA-C*1502.

21. The method of claim 1, wherein a combination of two or more alleles are detected.

22. A method for detecting an individual's decreased risk for type 1 diabetes comprising: detecting the presence of a protective class I HLA-A allele in a nucleic acid sample of the individual, wherein the presence of said allele indicates the individual's decreased risk for type 1 diabetes.

23. The method of claim 22, wherein the individual is Asian.

24. The method of claim 22, wherein the individual is a Filipino.

25. The method of claim 22, wherein the nucleic acid sample comprises DNA.

26. The method of claim 22, wherein the nucleic acid sample comprises RNA.

27. The method of claim 22, wherein the nucleic acid sample is amplified.

28. The method of claim 27, wherein the nucleic acid sample is amplified by a polymerase chain reaction.

29. The method of claim 22, wherein the allele is detected by amplification.

30. The method of claim 29, wherein the allele is detected by a polymerase chain reaction.

31. The method of claim 22, wherein the allele is detected by sequencing.

32. The method of claim 22, wherein the allele is detected by contacting the nucleic acid sample with one or more polynucleotides that hybridize under stringent hybridization conditions to one or more polymorphisms associated with said allele and detecting hybridization.

33. The method of claim 32, wherein the one or more polynucleotides are each individually complementary to a sequence found in exon 2 or exon 3 of a protective class I HLA-A allele.

34. The method of claim 32 wherein the one or more polynucleotides comprise at least one of the polynucleotide sequences listed in Table 9.

35. The method of claim 34, wherein the allele is the class I HLA-A*1101 allele.

36. The method of claim 22, wherein a combination of two or more alleles are detected.

37. A kit for determining an individual's risk for type 1 diabetes comprising:
(a) one or more polynucleotides each individually comprising a sequence that hybridizes under stringent hybridization conditions to a nucleic acid sequence in a type 1 diabetes-associated class I HLA-C allele, wherein said nucleic acid sequence comprises one or more polymorphisms associated with said allele; and
(b) instructions to use the kit to determine the individual's risk for type 1 diabetes.

38. The kit of claim 37, wherein one or more of the polynucleotides each individually comprise a sequence that is fully complementary to a nucleic acid sequence in a type 1 diabetes-associated class I HLA-C allele, wherein said nucleic acid sequence comprises one or more polymorphisms associated with said allele.

39. The kit of claim 37 or 38, further comprising sequencing primers.

40. The kit of claim 37 or 38, further comprising amplification primers.

41. The kit of claim 37 or 38, further comprising reagents for labeling one or more of the polynucleotides.

42. The kit of claim 37 or 38, wherein one or more of the polynucleotides are labeled.

43. The kit of claim 42 that includes one or more reagents to detect the label.

44. The kit of claim 37 or 38, wherein one or more of the nucleic acid molecules are each individually complementary to a polynucleotide sequence in a predisposing class I HLA-C allele.

45. The kit of claim 37 or 38, wherein one or more of the polynucleotides are each individually complementary to a polynucleotide sequence in exon 2 or exon 3 of a predisposing class I HLA-C allele.

46. The kit of claim 37 or 38, wherein one or more of the polynucleotides are each individually complementary to a polynucleotide sequence in a protective class I HLA-C allele.

47. The kit of claim 37 or 38, wherein one or more of the polynucleotides are each individually complementary to a polynucleotide sequence in exon 2 or exon 3 of a protective class I HLA-C allele.

48. The kit of claim 37 or 38, wherein one or more of the polynucleotides comprise at least one of the polynucleotide sequences listed in Table 7.

49. The kit of claim 48 wherein the allele is HLA-C*0102.

50. The kit of claim 37 or 38, wherein one or more of the polynucleotides comprise at least one of the polynucleotide sequences listed in Table 11.

51. The kit of claim 50 wherein the allele is HLA-C*1502.

52. The kit of claim 37 or 38, wherein said kit is configured to detect the presence of two or more type 1 diabetes-associated class I HLA-C alleles.

53. A kit for determining an individual's risk for type 1 diabetes comprising:
(a) one or more polynucleotides each individually comprising a sequence that hybridizes under stringent hybridization conditions to a nucleic acid sequence in a type 1 diabetes-associated class I HLA-A allele, wherein said nucleic acid sequence comprises one or more polymorphisms associated with said allele; and
(b) instructions to use the kit to determine the individual's risk for type 1 diabetes.

54. The kit of claim 53, wherein one or more of the polynucleotides each individually comprise a sequence that is fully complementary to a nucleic acid sequence in a type 1 diabetes-associated class I HLA-A allele, wherein said nucleic acid sequence comprises one or more polymorphisms associated with said allele.

55. The kit of claim 53 or 54, further comprising sequencing primers.

56. The kit of claim 53 or 54, further comprising amplification primers.

57. The kit of claim 53 or 54, further comprising reagents for labeling one or more of the nucleic acid molecules.

58. The kit of claim 53 or 54, wherein one or more of the polynucleotides is labeled.

59. The kit of claim 58, that includes one or more reagents to detect the label.

60. The kit of claim 53 or 54, wherein one or more of the polynucleotides are each individually complementary to a nucleic acid sequence in exon 2 or exon 3 of a protective class I HLA-A allele.

61. The kit of claim 53 or 54, wherein the one or more polynucleotides comprise at least one of the polynucleotide sequences listed in Table 9.

62. The kit of claim 61 wherein the allele is HLA-A*1101.

63. An array for determining an individual's risk for type 1 diabetes comprising one or more polynucleotides immobilized on a substrate, wherein each polynucleotide individually comprises a sequence that hybridizes under stringent hybridization conditions to a nucleic acid sequence in a type 1 diabetes-associated class I HLA-C allele, wherein said nucleic acid sequence comprises one or more polymorphisms associated with said allele.

64. The array of claim 63, wherein each polynucleotide individually comprises a sequence that is fully complementary to a nucleic acid sequence in a type 1 diabetes-associated class I HLA-C allele, wherein said nucleic acid sequence comprises one or more polymorphisms associated with said allele.

65. The array of claim 63 or 64, wherein one or more of the polynucleotides are labeled.

66. The array of claim 63 or 64, wherein one or more of the polynucleotide are each individually complementary to a polynucleotide sequence in a predisposing class I HLA-C allele.

67. The array of claim 63 or 64, wherein one or more of the polynucleotides are each individually complementary to a nucleic acid sequence in exon 2 or exon 3 of a predisposing class I HLA-C allele.

68. The array of claim 63 or 64, wherein one or more of the polynucleotides are each individually complementary to a nucleic acid sequence in a protective class I HLA-C allele.

69. The array of claim 63 or 64, wherein one or more of the polynucleotides are each individually complementary to a nucleic acid sequence in exon 2 or exon 3 of a protective class I HLA-C allele.

70. The array of claim 63 or 64, wherein one or more of the polynucleotides comprise at least one of the polynucleotide sequences listed in Table 7.

71. The array of claim 70 wherein the allele is HLA-C*0102.

72. The array of claim 63 or 64, wherein one or more of the polynucleotides comprise at least one of the polynucleotide sequences listed in Table 11.

73. The array of claim 72 wherein the allele is HLA-C*1502.

74. The array of claim 63 or 64, wherein said array is configured to detect the presence of two or more predisposing or protective HLA-C alleles or combinations of predisposing alleles, protective alleles or both.

75. A method for determining an individual's risk for type 1 diabetes comprising: detecting the presence of a type 1 diabetes-associated class I HLA-C allele in a nucleic acid sample of the individual by contacting the nucleic acid sample of the individual with the array of claim 64, wherein the presence of said allele indicates the individual's risk for type 1 diabetes.

76. An array for determining an individual's risk for type 1 diabetes comprising one or more polynucleotides immobilized on a substrate, wherein each polynucleotide individually comprises a sequence that hybridizes under stringent hybridization conditions to a nucleic acid sequence in a type 1 diabetes-associated class I HLA-A allele, wherein said nucleic acid sequence comprises one or more polymorphisms associated with said allele.

77. The array of claim 76, wherein each polynucleotide individually comprises a sequence that is fully complementary to a nucleic acid sequence in a type I diabetes-associated class I HLA-A allele, wherein said nucleic acid sequence comprises one or more polymorphisms associated with said allele.

78. The array of claim 76 or 77, wherein one or more of the polynucleotides are labeled.

79. The array of claim 76 or 77, wherein one or more of the polynucleotides are each individually complementary to a nucleic acid sequence in a protective class I HLA-A allele.

80. The array of claim 76 or 77, wherein one or more of the polynucleotides are each individually complementary to a nucleic acid sequence in exon 2 or exon 3 of a protective class I HLA-A allele.

81. The array of claim 76 or 77, wherein one or more of the polynucleotides comprise at least one of the sequences listed in Table 9.

82. The array of claim 81 wherein the protective class I HLA-A allele is HLA-A*101.

83. A method for determining an individual's risk for type 1 diabetes comprising: detecting the presence of a type 1 diabetes-associated class I HLA-A allele in a nucleic acid sample of the individual by contacting the nucleic acid sample of the individual with the array of claim 77, wherein the presence of said allele indicates the individual's risk for type 1 diabetes.

84. An array for determining an individual's risk for type 1 diabetes comprising one or more polynucleotides immobilized on a substrate that each individually comprises a polynucleotide sequence that hybridizes under stringent hybridization conditions to a nucleic acid sequence in a type 1 diabetes-associated class I HLA-A or -C allele comprising one or more polymorphisms associated with said allele, wherein the presence of two or more predisposing or protective HLA-A or -C alleles or combinations of predisposing alleles, protective alleles or both are detected.

85. The array of claim 84, wherein each polynucleotide individually comprises a sequence that is fully complementary to a nucleic acid sequence in a type 1 diabetes-associated class I HLA-A or -C allele, wherein said nucleic acid sequence comprises one or more polymorphisms associated with said allele.


SEQUENCE LISTING (34 sequences)

SEQ ID NO: 1 - DNA, 22 bp, Artificial
  Artificial Sequence Type Probe for Class I HLA-C Locus
ccggagtattgggaccgggaga

SEQ ID NO: 2 - DNA, 24 bp, Artificial
  Artificial Sequence Type Probe for Class I HLA-C Locus
gcctacgacgkcaaggattacatc

SEQ ID NO: 3 - DNA, 30 bp, Artificial
  Artificial Sequence Type Probe for HLA-A Locus
gagccgcgggcgccgtggatagagcaggag

SEQ ID NO: 4 - DNA, 30 bp, Artificial
  Artificial Sequence Type Probe for Class I HLA-A Locus
gaggacctgcgctcttggaccgcggcggac

SEQ ID NO: 5 - DNA, 21 bp, Artificial
  Artificial Sequence Type Probe for HLA-C Allele
gacacggatgtgaagaaatac

SEQ ID NO: 6 - DNA, 19 bp, Artificial
  Artificial Sequence Type Probe for HLA-C Allele
ctcccctctcggactcgcg

SEQ ID NO: 7 - DNA, 14 bp, Artificial
  Artificial Sequence Type Probe for HLA-C Allele
gccgcgggcgccgt

SEQ ID NO: 8 - DNA, 16 bp, Artificial
  Artificial Sequence Type Probe for HLA-C Allele
aggcacagactgaccg

SEQ ID NO: 9 - DNA, 16 bp, Artificial
  Artificial Sequence Type Probe for HLA-C Allele
agcctgcggaacctgc

SEQ ID NO: 10 - DNA, 18 bp, Artificial
  Artificial Sequence Type Probe for HLA-C Allel
ggcgtactggtcataccc

SEQ ID NO: 11 - DNA, 17 bp, Artificial
  Artificial Sequence Type Probe for HLA-C Allele
gcggagagcctacctgg

SEQ ID NO: 12 - DNA, 16 bp, Artificial
  Artificial Sequence Type Probe for HLA-C Allele
gagggcacgtgcgtgg

SEQ ID NO: 13 - DNA, 18 bp, Artificial
  Artificial Sequence Type Probe for HLA-C Allele
ctcaccggcctcgctctg

SEQ ID NO: 14 - DNA, 19 bp, Artificial
  Artificial Sequence Type Probe for HLA-C Allele
gggacacagcggtgtagaa

SEQ ID NO: 15 - DNA, 18 bp, Artificial
  Artificial Sequence Type Probe for HLA-C Allele
agccatacatcctctgga

SEQ ID NO: 16 - DNA, 18 bp, Artificial
  Artificial Sequence Type Probe for HLA-C Allele
gtatgaccagtccgccta

SEQ ID NO: 17 - DNA, 18 bp, Artificial
  Artificial Sequence Type Probe for HLA-C Allele
ggagcagctgagagccta

SEQ ID NO: 18 - DNA, 19 bp, Artificial
  Artificial Sequence Type Probe for HLA-C Allele
acacggcggtgtcgaaata

SEQ ID NO: 19 - DNA, 18 bp, Artificial
  Artificial Sequence Type Probe for HLA-C Allele
tcggtcagcctgtgcctg

SEQ ID NO: 20 - DNA, 17 bp, Artificial
  Artificial Sequence Type Probe for HLA-C Allele
agaggatgtctggctgc

SEQ ID NO: 21 - DNA, 18 bp, Artificial
  Artificial Sequence Type Probe for HLA-C Allele
gcgagtccaagaggggag

SEQ ID NO: 22 - DNA, 17 bp, Artificial
  Artificial Sequence Type Probe for HLA-C Allele
cgcagtttccgcaggtt

SEQ ID NO: 23 - DNA, 19 bp, Artificial
  Artificial Sequence Type Probe for HLA-C Allele
gtaggctaactggtcatgc

SEQ ID NO: 24 - DNA, 22 bp, Artificial
  Artificial Sequence Type Probe for HLA-A Allele
atgaggtatttctacacctccg

SEQ ID NO: 25 - DNA, 18 bp, Artificial
  Artificial Sequence Type Probe for HLA-A Allele
attgggaccaggagacac

SEQ ID NO: 26 - DNA, 21 bp, Artificial
  Artificial Sequence Type Probe for HLA-A Allele
ggtctgtgactgggccttcat

SEQ ID NO: 27 - DNA, 26 bp, Artificial
  Artificial Sequence Type Probe for HLA-A Allele
caggtccactcggtcaatctgtgact

SEQ ID NO: 28 - DNA, 20 bp, Artificial
  Artificial Sequence Type Probe for HLA-A Allele
ccatccagataatgtatggc

SEQ ID NO: 29 - DNA, 17 bp, Artificial
  Artificial Sequence Type Probe for HLA-A Allele
ggcgtcctgccggtacc

SEQ ID NO: 30 - DNA, 16 bp, Artificial
  Artificial Sequence Type Probe for HLA-A Allele
gaacgaggacctgcgc

SEQ ID NO: 31 - DNA, 18 bp, Artificial
  Artificial Sequence Type Probe for HLA-A Allele
acttgcgcttggtgatct

SEQ ID NO: 32 - DNA, 15 bp, Artificial
  Artificial Sequence Type Probe for HLA-A Allele
ggcccatgcggcgga

SEQ ID NO: 33 - DNA, 17 bp, Artificial
  Artificial Sequence Type Probe for HLA-A Allele
gagcagcagagagccta

SEQ ID NO: 34 - DNA, 13 bp, Artificial
  Artificial Sequence Type Probe for HLA-A Allele
gagggccggtgcg